(19) United States
(12) Patent Application Publication
(10) Pub. No.: US 2005/0262647 A1
(43) Pub. Date: Dec. 1, 2005

(54) Agent for dyeing fibers containing keratin

(76) Inventors: Horst Hoeffkes, Duesseldorf (DE); Doris Oberkobusch, Duesseldorf (DE); Hinrich Moeller, Monheim (DE)

Correspondence Address: HENKEL CORPORATION, THE TRIAD, SUITE 200, 2200 RENAISSANCE BLVD., GULPH MILLS, PA, 19406, US

(21) Appl. No.: 11/158,940

(22) Filed: Jun. 22, 2005

(30) Foreign Application Priority Data
Dec. 23, 2002 (DE) 102 61 656.6

(51) Int. Cl. A61K 7/13

(52) U.S. Cl. 8/405

(57) ABSTRACT

An agent for dyeing keratin-containing fibers, in particular, human hair, to give the fibers fashionable bright colors. The agent comprises A) an oxidizing agent, B) a CH-acidic compound and C) a reactive carbonyl compound. The invention also includes a process for dyeing keratin-containing fibers with the agent.

### CROSS REFERENCE TO RELATED APPLICATIONS

This application is a continuation under 35 U.S.C. § 365(c) and 35 U.S.C. § 120 of International application PCT/EP2003/014204, filed Dec. 13, 2003, incorporated herein by reference in its entirety. This application also claims priority under 35 U.S.C. § 119 of DE 102 61 656.6, filed Dec. 23, 2002, which is incorporated herein by reference in its entirety.

### BACKGROUND OF THE INVENTION

The invention relates to an agent, its use as well as a process for dyeing keratin-containing fibers, particularly human hair, the dyeing agent comprising an oxidizing agent, CH-acidic compounds and reactive carbonyl compounds.

For dyeing keratin-containing fibers, e.g., hair, wool or furs, generally either substantive dyes or oxidation dyes are used, the latter resulting from oxidative coupling of one or more developer components with each other or with one or more coupler components. Coupler and developer components are also referred to as oxidation dye precursors. For a small proportion of people, contact of the scalp with oxidation dye precursors may provoke an allergic reaction. Dyeing with a potential sensitization should therefore be substituted by dyeing, in which sensitization is excluded.

Young people in particular want especially bright, fashionable colors such as intense red, yellow, orange, green, blue or violet shades. Generally, the desired coloration can only be attained on very light hair, thus requiring a more or less strong lightening of the natural color shade.

Two processes are known from the state of the art for producing fashionable, intense color shades. In the first process, the coloration is made oxidatively by oxidizing agents, such as H₂O₂, and oxidation dye precursors (see Zviak, C., “Oxidation Colouring” in “The Science of Hair Care”, C. Zviak, Ed. (Marcel Dekker, New York, 1986), pages 263-286.

In the second process, substantive dyes (or substantives for short) are applied on pre-lightened hair or simultaneously with a bleaching agent. The application on pre-bleached hair, as for example disclosed in the published Patent EP-A1-920 856, is then a typical method, particularly if the dyes are unstable towards bleaching agents. The washing resistance of the colorations according to this second process is usually unsatisfactory.

Moreover, dyeing agents are known from the published Patent WO-A1-00/38638, which comprise a compound with a reactive carbonyl group in addition to a CH-acidic compound. A combined use of these components with an oxidizing agent is not the subject of this publication.

Substantive dyes are usually added for shading oxidation colorations. The former are characterized, as discussed earlier, by poor wash fastness, whereby the shades created by the oxidation dyeing with substantives can be removed by hair washing. The resulting costly and time-consuming re-coloration over short periods should be avoided if possible. With this aim, the coloration should permit a color life of at least 10 hair washes.

### BRIEF SUMMARY OF THE INVENTION

The object of the present invention is to provide an agent as well as a process for dyeing keratin-containing fibers, whereby the fibers acquire fashionable, bright colors, together with a good resistance to washing of the coloration and thereby, also in combination with oxidation hair dyeing agents, a long-lived shading is obtained.

Surprisingly, it has now been found that fashionable and bright color tints can be produced on keratin-containing fibers when a dyeing agent comprising an oxidizing agent (hereafter component A), CH-acidic compounds with the Formulae C1-C22 illustrated below (hereafter component B) and at least one reactive carbonyl compound (hereafter component C), particularly selected from compounds according to Formulae I, IV, V, Vla and Vlb, is applied onto the fibers. Wash-resistant colorations are obtained, which enable a sustained shading of oxidation colorations. Sensitization of the scalp can be avoided by the use of this dyeing agent.

### DETAILED DESCRIPTION OF CERTAIN EMBODIMENTS OF THE INVENTION

In general, CH-acids are recognized as compounds that possess a hydrogen atom bonded to an aliphatic carbon atom, in which the carbon-hydrogen bond is activated due to the electron-withdrawing substituents. According to the invention, CH-acidic compounds also include enamines, which result from the alkaline treatment of quaternized N-heterocycles having a CH-acidic alkyl group that is conjugated with the quaternary nitrogen.

According to the invention, reactive carbonyl compounds possess at least one carbonyl group as the reactive group, which reacts with the CH-acidic compounds to form a carbon-carbon bond. Moreover, according to the invention, such compounds are also applicable as component C, in which the reactive carbonyl group is protected or derivatized in such a manner that the reactivity of the carbon atom of the derivatized or protected carbonyl group remains with respect to the inventive CH-acidic compounds. These derivatives are preferably condensation compounds from the reaction of the carbonyl group of the reactive carbonyl compound with amines and their derivatives forming imines or oximes as the condensation compounds from alcohols forming acetals or ketals as the condensation compounds.

Accordingly, a first subject of the invention is an agent for dyeing keratin-containing fibers, particularly human hair, characterized in that it comprises

(A) an oxidizing agent,

(B) at least one CH— acidic compound selected from compounds according to the following Formulae C1-C22:

compounds with the Formula C1
M¹—CH₂—M² (C1)
in which
M¹ is a group —C(O)M³, —C(O)OM³, —S(O)M³, —SO₂M³, in which M³ stands for a hydrogen atom, an aryl group or a C₁-C₆ alkyl group, or means a group —C(M⁴)═C(C≡N)₂, in which M⁴ stands for a hydrogen atom, a C₁-C₄ alkyl group or an aryl group,
M² has the same meaning as M¹ or is a cyanide group, a substituted or unsubstituted aryl group or aryl-C₁-C₄ alkyl group, a substituted or unsubstituted, saturated or unsaturated heterocycle,
   compounds with the Formula C2 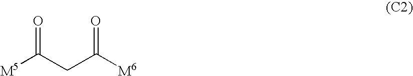
   in which
M⁵ is a cyanide group, a substituted or unsubstituted aryl group or an aryl-C₁-C₄ alkyl group, a substituted or unsubstituted, saturated or unsaturated heterocycle or a group —C—O—M⁷ or C(O)OM⁷, in which M⁷ stands for a hydrogen atom or a C₁-C₆ alkyl group,
M⁶ is a substituted or unsubstituted C₁-C₆ alkyl group, an acetyloxy group, a C₃-C₆ cycloalkyl group, a substituted or unsubstituted aryl group or aryl-C₁-C₄ alkyl group, a substituted or unsubstituted aminoaryl group, a substituted or unsubstituted, saturated or unsaturated heterocycle,
   compounds with the Formula C3 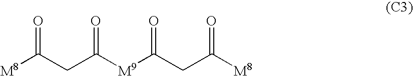
   in which
M⁸ is a cyanide group, a substituted or unsubstituted aryl group or aryl-C₁-C₄ alkyl group, a substituted or unsubstituted, saturated or unsaturated heterocycle or a group —COM¹⁰ or COOM¹⁰, in which M¹⁰ stands for a hydrogen atom or a C₁-C₆ alkyl group,
M⁹ is a substituted or unsubstituted aryl group or aryl-C₁-C₄ alkyl group, a substituted or unsubstituted aminoaryl group, a substituted or unsubstituted, saturated or unsaturated heterocycle,
   pyrazole derivatives (a): selected from the following Formulae C4 and C5 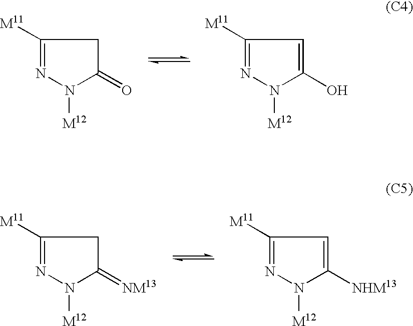
   in which
M¹¹ and M¹² independently of one another stand for a substituted or unsubstituted C₁-C₆ alkyl group, a C₃-C₆ cycloalkyl group, a substituted or unsubstituted aryl group or aryl-C₁-C₄ alkyl group, a substituted or unsubstituted aminoaryl group, a substituted or unsubstituted, saturated or unsaturated heterocycle, wherein M¹¹ can additionally mean a C₁-C₆ alkoxy group,
M¹³ is a hydrogen atom or a substituted or unsubstituted C₁-C₆ alkyl group,
   (b): two pyrazole rings with the Formulae C4 or C5 linked through M¹¹ or M¹²
   barbituric acid derivatives with the Formula C6 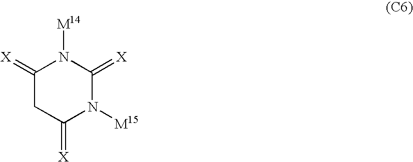
   in which
M¹⁴ and M¹⁵ independently of one another stand for a C₁-C₆ alkyl group, a C₂-C₆ alkenyl group, a C₃-C₆ cycloalkyl group, an aryl-C₁-C₄ alkyl group, a substituted or unsubstituted aryl group or a bicyclic group linked through the radicals M¹⁴ or M¹⁵,
X stands for an oxygen or a sulfur atom,
   pyridine derivatives with the Formulae C7a and C7b 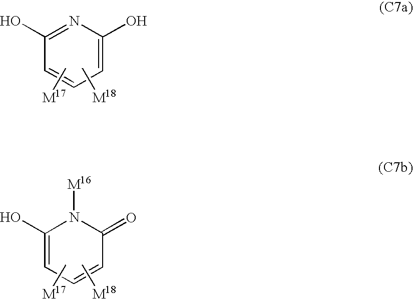
   in which
M¹⁶ is a substituted or unsubstituted C₁-C₆ alkyl group or substituted or unsubstituted aryl group,
M¹⁷ is a hydrogen atom, a substituted or unsubstituted C₁-C₆ alkyl group or a substituted or unsubstituted aryl group,
M¹⁸ is a hydrogen atom, a cyanide group, a substituted or unsubstituted C₁-C₆ alkyl group, a group COOM¹⁹, wherein M¹⁹ means a hydrogen atom or a substituted or unsubstituted C₁-C₆ alkyl group,
   Compounds with the Formula C8 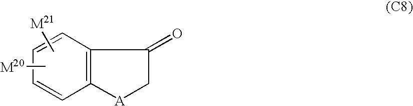
   in which
A stands for an oxygen atom, a sulfur atom, a sulfoxyl group, a sulfonyl group or a group NM^{20a}, wherein M^{20a} means a hydrogen atom, a substituted or unsubstituted C₁-C₆ alkyl group,
M²⁰ and M²¹ stand independently from one another for a hydrogen-, a fluorine, chlorine, a bromine atom, a hydroxyl group, a nitro group, a C₁-C₆ alkyl group, a C₁-C₆ alkoxy, a carboxamide, a sulfonamide, a carboxylic, a C₁-C₄ acyl, a cyanide group or an amino group —NM²²M²³, in which the M²² and M²³ independently of one another stand for a hydrogen atom or a C₁-C₆ alkyl group,
   compounds with the Formulae C₉ and C₁₀ 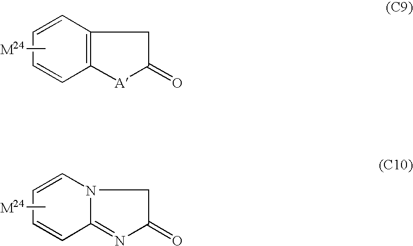
   in which
A′ stands for an oxygen atom, a sulfur atom or a group NM²⁵, in which M²⁵ means a hydrogen atom, a substituted or unsubstituted C₁-C₆ alkyl group,
M²⁴ stands for a hydrogen-, fluorine-, chlorine-, a bromine atom, a hydroxyl group, a nitro group, a C₁-C₆ alkyl, a C₁-C₆ alkoxy, a carboxamide, a sulfonamide, a carboxylic, a C₁-C₄ acyl, a cyanide group or an amino group —NM²⁶M²⁷, in which M²⁶ and M²⁷ independently of one another stand for a hydrogen and a C₁-C₆ alkyl group,
   compounds with the Formula C11 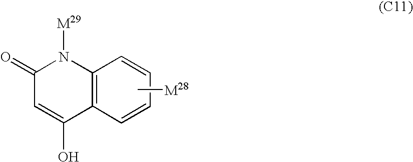
   in which
M²⁸ is a hydrogen atom, a halogen atom, a hydroxyl group, a substituted or unsubstituted C₁-C₆ alkyl group, a C₁-C₆ alkoxy group or a nitro group,
M²⁹ stands for a hydrogen atom or a C₁-C₄ alkyl group
   indanedione derivatives with the Formula C12 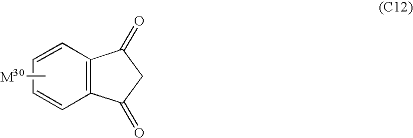
   in which
M³⁰ is a hydrogen-, fluorine-, chlorine-, a bromine atom, a nitro group, a C₁-C₆ alkyl, a C₁-C₆ alkoxy, a carboxamide, a sulfonamide or a cyanide group
   compounds with the Formula C13 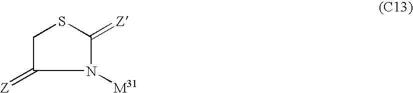
   in which
Z stands for an oxygen atom or a group NM³², in which M³² means a hydrogen atom or a C₁-C₆ alkyl group,
Z′ stands for a sulfur atom or a group NM³³, in which M³³ means a hydrogen atom or a C₁-C₆ alkyl group,
M³¹stands for a hydrogen atom, a C₁-C₆ alkyl group or a C₁-C₄ carboxyalkyl group,
   dioxopyrazole compounds with the Formula C14 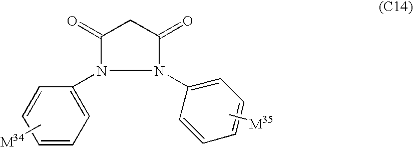
   in which
M³⁴ and M³⁵ independently of one another stand for a hydrogen-, fluorine-, chlorine-, a bromine atom, a hydroxyl group, a nitro group, a C₁-C₆ alkyl, a C₁-C₆ alkoxy, a carboxamide, a sulfonamide, a carboxylic, a C₁-C₄ acyl, a cyanide group or an amino group —NM³⁶M³⁷, in which M³⁶ and M³⁷ independently of one another stand for a hydrogen or a C₁-C₆ alkyl group,
   5-oxoimidazole derivatives with the Formula C15 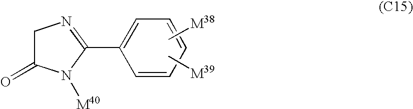
   in which
M³⁸ and M³⁹ independently of one another stand for a hydrogen-, fluorine-, chlorine-, a bromine atom, a hydroxyl group, a nitro group, a C₁-C₆ alkyl, a C₁-C₆ alkoxy, a carboxamide, a sulfonamide, a carboxylic, a C₁-C₄ acyl, a cyanide group or an amino group —NM⁴¹ M⁴², in which M⁴¹ and M⁴² independently of one another stand for a hydrogen atom or a C₁-C₆ alkyl group,
M⁴⁰ stands for a hydrogen atom or a C₁-C₆ alkyl group,
   derivatives of dehydrobutyrolactone with the Formula C16 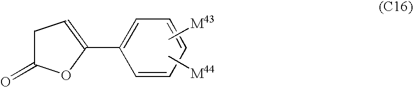
   in which
M⁴³ and M⁴⁴ independently of one another stand for a hydrogen-, fluorine-, chlorine-, a bromine atom, a hydroxyl group, a nitro group, a C₁-C₆ alkyl, a C₁-C₆ alkoxy, a carboxamide, a sulfonamide, a carboxylic, a C₁-C₄ acyl, a cyanide group or an amino group —NM⁴⁵M⁴⁶, in which M⁴⁵ and M⁴⁶ independently of one another stand for a hydrogen atom or a C₁-C₆ alkyl group, compounds with the Formula C17 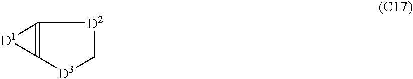
   in which
D¹ is a condensed aromatic or heteroatomic ring,
D² is a carbonyl group, a group C═CD^{I}D^{II} or a group CD^{I}D^{II}, in which D^{I} or D^{II} each have a substituent with a Hammett Constant between 0.4 and 2.0, or both substituents add up to a Hammett Constant between 0.4 and 2.0;
D³ stands for a carbonyl group, an oxygen atom, a sulfur atom, a group NM⁴⁷, when D² is not oxygen, or a group C═S, a group C═NR⁴⁸, a sulfinyl group, a sulfonyl group, wherein R⁴⁷ and R⁴⁸ independently of one another mean a hydrogen atom or a C₁-C₄ alkyl radical,
   pyrimidine derivatives with the Formula C18 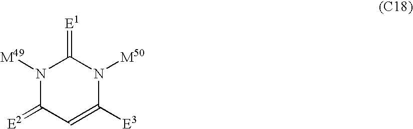
   in which
M⁴⁹ and M⁵⁰ independently of one another are a hydrogen atom or a substituted or unsubstituted C₁-C₆ alkyl group,
E¹ stands for an oxygen, a sulfur atom or a NH group,
E² stands for a NH group or an oxygen atom,
E³ stands for an amino group or a hydroxyl group,
   with the proviso, that
   a) when E¹ and E² stand for an oxygen atom, E³ is not a hydroxyl group, and
   b) when E¹ is a sulfur atom and E² is an oxygen atom, E³ is not a hydroxyl group,
   quaternized nitrogen compounds of Formula C19 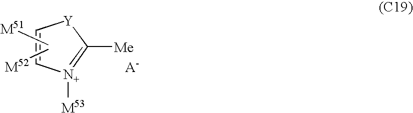
   in which,
M⁵¹ and M⁵² independently of one another stand for a hydrogen atom, a halogen atom, a hydroxyl group, a C₁-C₄ hydroxyalkyl group, a C₁-C₆ aminoalkyl group, a C₁-C₄ dialkylamino-C₁-C₄ alkyl group, a linear or branched C₁-C₆ alkyl group, a C₂-C₆ alkenyl group, an optionally substituted aryl group, a sulfonic acid group, a carboxylic group, a formyl group, a nitro group, a cyanide group or a group —NM⁵⁴M⁵⁵, wherein M⁵⁴ and M⁵⁵ independently of one another stand for a hydrogen atom, a C₁-C₆ alkyl group, a C₂-C₆ alkenyl group, an aryl C₁-C₄ alkyl group, or a C₁-C₄ hydroxyalkyl group, wherein M⁵¹ and M⁵² can together form a condensed 5- or 6-membered aliphatic or aromatic or heteroaromatic ring, which is again substituted with the radicals M⁵⁶ and M⁵⁷, wherein M⁵⁶ and M⁵7 independently of one another stand for the radicals defined for M⁵¹,
M⁵³ stands for a hydrogen atom, a C₁-C₄ hydroxyalkyl group, a C₁-C₆ aminoalkyl group, a C₁-C₄ dialkylamino-C₁-C₄ alkyl group, a linear or branched C₁-C₆ alkyl group, a C₂-C₆ alkenyl group, an optionally substituted aryl group, a C₁-C₄ sulfoalkyl group, a C₁-C₄ carboxyalkyl group or a C₂-C₆ polyhydroxyalkyl group,
Y stands for an oxygen atom, a sulfur atom, an optionally substituted methylene group or a group NM⁶⁰, wherein M⁶⁰ can stand for the same groups that are defined for M⁵⁵,
A⁻ stands for a chloride, bromide, iodide, hexafluorophosphate, tetrachlorozincate, tetrafluoroborate, trifluoromethyl sulfonate, methyl sulfonate or p-toluene sulfonate,
   onium compounds with the Formulae C20 and C21 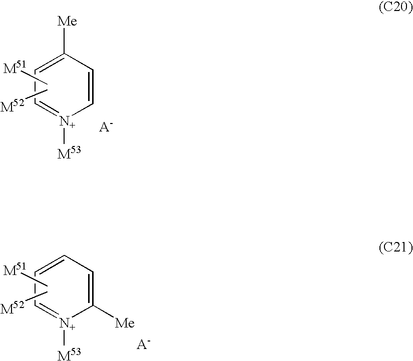
   wherein M⁵¹, M⁵², M⁵³ and A⁻ are selected from the groups that are defined for compound C19.
   compounds with the general Formula C22 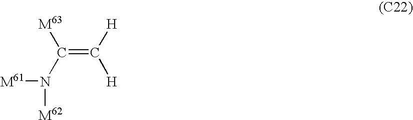
   in which
M⁶¹ stands for an aromatic radical, particularly for a 5- or 6-membered aryl radical optionally substituted with a C₁-C₄ alkyl, C₁-C₄ hydroxyalkyl, hydroxy, methoxy or halide group, preferably a phenyl radical, or a 5- or 6-membered, condensed, aliphatic or aromatic, carbocyclic or heterocyclic ring preferably a phenyl radical, a quinoline radical or a pyridyl radical.
M⁶² stands for a hydrogen atom, a linear or branched C₁-C₈ alkyl group, a linear or branched C₁-C₈ hydroxyalkyl group or a C₁-C₈ alkoxyalkyl group, wherein an oxygen atom can be between the C atoms of the alkyl chain, and M⁶³ stands for a linear or branched C₁-C₈ alkyl group, a C₁-C₆ alkoxyalkyl C₁-C₆ alkyl group, a C₁-C₆ alkylamino C₁-C₆ alkyl group, a C₁-C₆ alkylmercapto C₁-C₆ alkyl group, a C₁-C₆ alkoxy C₁-C₆ alkylene group, a C₁-C₆ alkylamino C₁-C₆ alkylene group, a C₁-C₆ alkylmercapto C₁-C₆ alkylene group, a straight chain or branched C₁-C₈ alkylene group or an oxygen atom, a nitrogen atom or a sulfur atom, with the proviso that the radicals M⁶¹ and M⁶³, together with the nitrogen atom and the carbon atom of the basic enamine structure, form a cyclic compound, when M⁶³ equals a linear or branched C₁-C₈ alkylene group, a C₁-C₈ alkoxyalkylene group, a C₁-C₆ alkylamino C₁-C₆ alkylene group, a C₁-C₆ alkylmercapto C₁-C₆ alkylene group, an oxygen atom, a nitrogen atom or a sulfur atom, wherein preferably M⁶³ is bonded to the aromatic radical M⁶¹ with the carbon that stands in the ortho-position to the enamine substituted carbon,
   (C) at least one reactive carbonyl compound.

Particularly suitable oxidizing agents of component A for human hair are preferably H₂O₂, H₂O₂ generating compounds or combinations of these, in which these compounds may also be loaded on an inert carrier. H₂O₂ can be used, e.g., in combination with Na—, K—, NH₄— peroxydisulfate or the corresponding peroxydiphosphonates in a slightly acidic to alkaline medium, particularly between pH 5.0 to 10.5. Compounds such as e.g. magnesium peroxide can also be added to the H₂O₂. H₂O₂ can be added to the oxidizing agents in free form or combined as H₂O₂-generating compounds, e.g., as sodium carbonate peroxyhydrate, urea peroxide or melamine perhydrate. The oxidizing agents are preferably added in a quantity of 0.01 to 6 wt. %, based on the applied solution.

The CH— acidic compounds of component B with the Formulae Cl, C2 and C3 are preferably selected from benzoylacetonitrile, malonic acid, esters of malonic acid and their derivatives as well as acetoacetic acid, esters of acetoacetic acid and their derivatives.

Examples of CH— acidic compounds of component B with the Formulae C4 and C5 are pyrazol-5-one, 3-methyl-pyrazol-5-one, 1-phenyl-3-methyl-pyrazol-5-one, 1-(β-cyanoethyl)-3-methyl-pyrazol-5-one, 1,3-dimethyl-pyrazol-5-one, 1-(β-acetoxyethyl)-3-methyl-pyrazol-5-one, 1-(o-chlorophenyl)-3-methyl-pyrazol-5-one, 1-phenyl-3-carbomethoxy-pyrazol-5-one, 1-(3-aminophenyl-pyrazol-5-one, 1-(4-aminophenyl)-pyrazol-5-one, 3-methyl-pyrazol-5-one-1-carboxamide, 1-phenyl-pyrazol-5-one-3-carboxamide, 1-phenyl-5-amino-pyrazol, 1-benzyl-5-amino-pyrazole, 1-cyclohexyl-5-amino-pyrazole, 1-ethyl-3-methyl-5-amino-pyrazole, 1-benzyl-3-phenyl-5-amino-pyrazole, 1-isopentyl-5-aminopyrazole, 1-furfuryl-5-aminopyrazole, 2-methyl-4H-pyrazolo(5)-[2,3-a]-benzimidazole, [1-(3-thiacyclopentyl)-3-methyl-pyrazol-5-one-S-dioxide], 2-methyl-1H-3,3a,8-triazacyclopenta[a]indene.

The barbituric acid derivatives with the Formula C6 are preferably selected from di-n-butylbarbituric acid, di-iso-butyl-barbituric acid, di-N-amylbarbituric acid, di-iso-amylbarbituric acid, di-n-hexylbarbituric acid, di-benzylbarbituric acid, di-β-phenylethylbarbituric acid, di-cyclohexylbarbituric acid, di-phenylbarbituric acid, di-p-tolylbarbituric acid, di-p-methoxybenzylbarbituric acid, N-methyl-N′-n-butylbarbituric acid, N-methyl-N′-benzylbarbituric acid, N-methyl-N′-β-phenylethylbarbituric acid, N-methyl-N′-γ-phenylpropylbarbituric acid, N-methyl-N′-γ-phenylbutylbarbituric acid, N-methyl-N′-α-isobutyl-γ-phenylpropylbarbituric acid, N-methyl-N′-cyclohexylbarbituric acid, N-methyl-N′-phenylbarbituric acid, N-methyl-N′-p-toluylbarbituric acid, N-methyl-N′-norbornylmethylbarbituric acid as well as the corresponding N-ethyl-and N- butyl derivatives of the above.

Preferred examples of pyridine derivatives with Formula C7a or C7b are 2,6-dihydroxy-3-cyan-4-methyl-pyridin, cyanopyridone, aminonitropyridone and aminocyanopyridone, particularly N-methyl-3-cyano-4-methyl-6-hydroxypyridone-2, N-ethyl-3-cyano-4-methyl-6-hydroxy-pyridone-2, N-β-methoxyethyl-3-cyano-4-methyl-6-hydroxypyridone-2, 2,6-dihydroxy-3-cyano-4-methylpyridine, N-β-hydroxyethyl-3-cyano-4-methyl-6-hydroxypyridone-2, N-butyl-3-cyano-4-methyl-6-hydroxypyridone-2 und N-phenyl-3-cyano-4-methyl-6-hydroxypyridone-2.

Compounds with Formula C8 may be selected from 6-hydroxybezofuran-(2H)-one or benzofuran-(2H)-one.

CH— acidic compounds with the Formula C9 may be selected from 1,3-dihydro-indol-2-one, 3H-benzofuran-2-one (2-cumaranone), 1-methyl-1,3-dihydro-indol-2-one, 5-methoxy-3H-benzofuran-2-one, 5-nitro-1,3-dihydroindol-2-one, 1-methyl-5-nitro-1,3-dihydroindol-2-one, 6-methoxy-1,3-dihydroindol-2-one, 5-chloro-1,3-dihydroindol-2-one, 5,6-difluoro-1,3-dihydroindol-2-one, 6-hydroxy-5-methoxy-1,3-dihydroindol-2-one, 5,6-dimethoxy-1,3-dihydroindol-2-one and 6-trifluoromethyl-1,3-dihydroindol-2-one.

Examples of CH-acidic compounds with the Formula C10 are imidazo[1,2-a]pyridin-2-one and 6-bromoimidazo[1,2-a]pyridin-2-one.

The CH-acidic compounds with Formula C11 are preferably selected from those in which a hydrogen atom stands for M²⁹ in Formula C11, such as 2,4-dihydroxyquinoline.

A suitable example of an indane derivative of Formula C12 is 1,3-indanedione.

The CH— acidic compounds of Formula C13 are preferably selected from rhodamine and 2-amino-4-imino-2-thiazoline.

The preferred CH— acidic compound with Formula C14 may be cited as 1,2-diphenyl-3,5-dioxopyrazole.

The CH— acidic compounds of Formula C15 are preferably selected from 2-phenyl-3,5-dihydroimidazol-4-one and 3-methyl-2-p-toluyl-3,5-dihydroimidazol-4-one.

A preferred example of a CH— acidic compound with Formula C16 is phenyidihydrobutyrolactone.

The CH— acidic compounds of Formula C17 are preferably selected from 1,1-dioxo-1,2-dihydro-11,6-benzo[b]-thiophen-3-one and 2-(1,1-dioxo-1,2-dihydro-11,6-benzo[b]-thiophen-3-ylidene)malonitrile.

Examples of pyrimidine derivatives with Formula C18 are 2,4-diamino-6-hydroxypyrimidine, 2,6-diamino-4-hydroxypyrimidine, 4-amino-2,6-dihydroxypyrimidine, 4-amino-6-hydroxy-2 sulfanylpyrimidine, 2-amino-4,6-dihydroxypyrimidine, 4,6-dihydroxy-1-mercaptopyrimidine and 2,4,6-trihydroxypyrimidine; 2,4-diamino-6-hydroxypyrimidine being particularly preferred.

Examples of preferred compounds used with the Formula C19 may be cited as: 1,2, 3,3-tetramethyl-3H-indolium-, 2,3-dimethylbenzothiazolium-, 2,3-dimethyl-6-nitrobenzothiazolium-, 3-benzyl-2-benzothiazolium-, 2-methyl-3-propylbenzo- thiazolium-, 2,4-dimethyl-3-ethylthiazolium-, 3-(2-carboxyethyl)-2,5-dimethylbenzothiazolium-, 1,2,3-trimethylbenzimidazolium-, 5,6-dichloro-1,3-diethyl-2-methylbenzimidazolium-, 3-ethyl-2-methylbenzothiazolium-, 3-ethyl-2-methylnaphtho[1,2-d]thiazolium-, 5-chloro-3-ethyl-2-methylbenzothiazolium-, 3-ethyl-2-methylbenzoxazolium-salts, which can be, e.g., chlorides, bromides, iodides, methanesulfonates, benzenesulfonates, p-toluenesulfonates, trifluoromethanesulfonates, methylsulfates, tetrafluoroborates, as well as 2-methyl-3-(3-sulfopropyl)benzothiazolium hydroxide, inner salt, 4-methyl-1-(3- sulfopropyl)pyridinium hydroxide, inner salt, 4-methyl-1-(3-sulfopropyl)-quinolinium hydroxide, inner salt, 5-methoxy-2-methyl-3-(3-sulfopropyl)-benzothiazolium hydroxide, inner salt and any mixtures of the above compounds.

Examples of preferred, used compounds according to Formulae C20 and C21 are: 1,4-dimethylquinolinium-, 1,2-dimethylquinolinium-, 1,4-dimethylpyridinium-, 1,2-dimethylpyridinium-, 2,4,6-trimethylpyrilium-, 2-methyl-1-ethylquinolinium-, 2,3-dimethylisoquinolinium-salts, e.g., the chlorides, bromides, iodides, methanesulfonates, benzenesulfonates, p-toluenesulfonates, trifluoromethanesulfonates, methylsulfates, tetrafluoroborates and any mixtures of the above compounds.

The CH-acidic compounds are particularly preferably selected from benzofuran-(2H)-one, benzoylacetonitrile and 2-amino-4-imino-2-thiazoline and from their physiologically acceptable salts.

The CH-acidic compounds are each preferably used in an amount of 0.03 to 65 mmol, particularly 1 to 40 mmol, based on 100 g of the added finished dyeing agent.

The reactive carbonyl compounds of component C are preferably selected from the group, which is formed from compounds according to Formulae I, IV, V, Vla and Vlb, 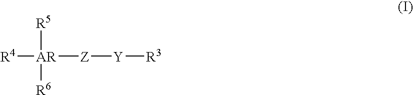
wherein
AR stands for benzene, naphthalene, pyridine, pyrimidine, pyrazine, pyridazine, carbazole, pyrrole, pyrazole, furan, thiophene, 1,2,3-triazine, 1,3,5-triazine, quinoline, isoquinoline, indole, indoline, indolizine, indane, imidazole, 1,2,4-triazole, 1,2,3-triazole, tetrazole, benzimidazole, 1,3-thiazole, benzothiazole, indazole, benzoxazole, quinoxaline, quinazoline, quinolizine, cinnoline, acridine, julolidine, acenaphthene, fluorene, biphenyl, diphenylmethane, benzophenone, diphenyl ether, azobenzene, chromone, coumarine, diphenylamine, stilbene, wherein the N-heteroaromatics can also be quaternized,
R³ stands for a hydrogen atom, a C₁-C₆ alkyl, C₂-C₆ acyl, C₂-C₄ alkenyl, C₁-C₄ perfluoroalkyl, an optionally substituted aryl or heteroaryl group,
R⁴, R⁵ and R⁶ independently from one another stand for a hydrogen atom, a halogen atom, a C₁-C₆ alkyl, C₁-C₆ alkoxy, C₁-C₆ aminoalkyl, C₁-C₆ hydroxyalkyl group, a C₁-C₆ alkoxy-C₁-C₆ alkyloxy group, a C₂-C₆ acyl group, an acetyl, a carboxyl, carboxylato, carbamoyl, sulfo, sulfato, sulfonamide, sulfonamido, C₂-C₆ alkenyl, an aryl, an aryl C₁-C₆ alkyl group, a hydroxyl, a nitro, a pyrrolidino, a morpholino, a piperidino, an amino or ammonio or a 1-imidazol(in)io group, wherein the last three groups can be substituted with one or more C₁-C₆ alkyl-, C₁-C₆ carboxyalkyl-, C₁-C₆ hydroxyalkyl, C₂-C₆ alkenyl, C₁-C₆ alkoxy-C₁-C₆ alkyl, with optionally substituted benzyl groups, with sulfo-(C₁-C₄)-alkyl or heterocycle-(C₁-C₄) alkyl groups, wherein also two of the radicals R⁴, R⁵, R⁶ and —Z—Y—R³, together with the remainder of the molecule, can form a condensed, optionally substituted 5-, 6- or 7-membered ring that can equally have a condensed aromatic ring on it, wherein the system AR, depending on the ring size, can have further substituents that independently of one another can stand for the same groups as R⁴, R⁵ and R⁶,
Z stands for a direct bond, a carbonyl, a carboxy-(C₁-C₄) alkylene, an optionally substituted C₂-C₆ alkenylene, C₄-C₆ alkadienylene, furylene, thienylene, arylene, vinylenearylene, vinylenefurylene, vinylenethienylene group, wherein Z, together with the —Y—R³ group can also form an optionally substituted 5-, 6- or 7-membered ring,
Y stands for a group selected from carbonyl, a group according to Formula II and a group according to Formula III, 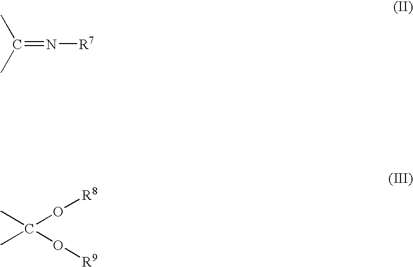
   wherein
R⁷ stands for a hydrogen atom, a hydroxyl group, a C₁-C₄ alkoxy group, a C₁-C₆ alkyl group, a C₁-C₆ hydroxyalkyl group, a C₂-C₆ polyhydroxyalkyl group, a C₁-C₆ alkoxy-C₁-C₆ alkyl group,
R⁸ and R⁹ independently of one another stand for a C₁-C₆ alkyl group, an aryl group or form, together with the structural element O—C—O of Formula III, a 5- or 6-membered ring,
   aldehydes with the Formula (IV) 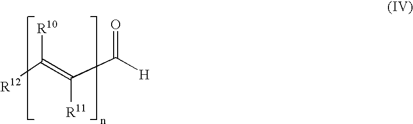
   in which
R¹⁰ and R¹¹ independently of one another stand for a hydrogen atom, a C₁-C₆ alkyl group, a C₁-C₄ hydroxyalkyl group, a C₂-C₈ polyhydroxyalkyl group, a C₁-C₄ alkoxy C₁-C₄ alkyl group, a C₁-C₄ alkoxy group, a group —OCF₃ or a group —CF₃, wherein the radicals R¹⁰ and R¹¹, together with the vinylene moiety in the brackets, can form a 5- or 6-membered, aromatic or aliphatic, carbocyclic or heterocyclic ring,
n equals 0, 1, 2 or 3
R¹² stands for a hydrogen atom, a C₁-C₆ alkyl group, a C₁-C₄ hydroxyalkyl group, a C₂-C₈ polyhydroxyalkyl group, a C₁-C₄ alkoxy C₁-C₄ alkyl group, a C₁-C₄ alkoxy group, a group —OCF₃ or a group —CF₃,
   ketones with the Formula (V) 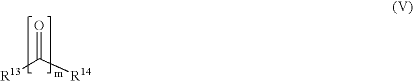
   in which
R¹³ stands for a hydrogen atom, a C₁-C₆ alkyl group, a C₁-C₄ hydroxyalkyl group, a C₂-C₈ polyhydroxyalkyl group, a C₁-C₄ alkoxy C₁-C₄ alkyl group, a C₁-C₄ alkoxy group, a group —OCF₃ or a group —CF₃,
R¹⁴ stands for a C₁-C₆ alkyl group, a C₁-C₄ monohydroxyalkyl group, a C₂-C₈ polyhydroxyalkyl group,
m equals 1 or 2
   wherein R¹³ and R¹⁴, together with the carbonyl moiety can form a 5- or 6-membered, aliphatic or aromatic, carbocyclic or heterocyclic ring that optionally again carries a condensed 5- or 6-membered, aliphatic or aromatic, carbocyclic or heterocyclic ring, with the proviso that each heterocyclic ring comprises nitrogen or sulfur atoms as the heteroatoms,
   diiminoisoindoline- or 3-aminoisoindoline derivatives according to Formulae Vla and Vlb, 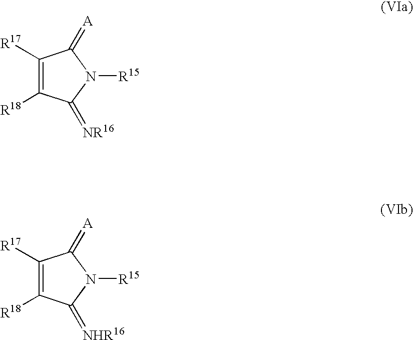
   in which
A stands for an oxygen atom or a NH group,
R¹⁵ and R¹⁶ independently of one another stand for a hydrogen atom, a C₁-C₄ alkyl group, a C₁-C₄ hydroxyalkyl group, a C₂-C₆ polyhydroxyalkyl group, a group R^{I}R^{II}N—(CH₂)_{q}—, wherein R^{I} and R^{II} independently of one another stand for a C₁-C₆ alkyl group or an aryl C₁-C₆ alkyl group and q stands for a number 1, 2, 3, 4, 5 or 6, wherein R^{I} and R^{II}, together with the nitrogen atom, can form an aromatic or aliphatic 5-, 6- or 7-membered heterocycle,
R¹⁷ and R¹⁸, together with the remainder of the molecule, can form a 5- or 6-membered, aromatic or heteroaromatic ring that can optionally be substituted.

When R¹⁵ stands for a hydrogen atom, then the compound according to Formula Vla or Vlb exists in chemical equilibrium with each corresponding tautomer.

According to a special embodiment, it is preferred to select compounds for component C from the group consisting of:

5-(4-dimethylaminophenyl)penta-2,4-dienal, 5-(4-diethylaminophenyl)penta-2,4-dienal, 5-(4-methoxyphenyl)penta-2,4-dienal, 5-(3,4-dimethoxyphenyl)penta-2,4-dienal, 5-(2,4-dimethoxyphenyl)penta-2,4-dienal, 5-(4-piperidinophenyl)penta-2,4-dienal, 5-(4-morpholinophenyl)penta-2,4-dienal, 5-(4-pyrrolidinophenyl)penta-2,4-dienal, 6-(4-dimethylaminophenyl)hexa-3,5-dien-2-one, 6-(4-diethylaminophenyl)hexa-3,5-dien-2-one, 6-(4-methoxyphenyl)hexa-3,5-dien-2-one, 6-(3,4-dimethoxyphenyl)hexa-3,5-dien-2-one, 6-(2,4-dimethoxyphenyl)hexa-3,5-dien-2-one, 6-(4-piperidinophenyl)hexa-3,5-dien-2-one, 6-(4-morpholinophenyl)hexa-3,5-dien-2-one, 6-(4-pyrrolidinophenyl)hexa-3,5-dien-2-one, 5-(4-dimethylaminonaphth-1-yl)penta-2,4-dienal,

2-nitropiperonal, 5-nitropiperonal, 6-nitropiperonal, 5-hydroxy-2-nitropiperonal, 2-hydroxy-5-nitropiperonal, 2-chloro-6-nitropiperonal, 5-chloro-2-nitropiperonal, 2,6-dinitropiperonal,

2-nitrobenzaldehyde, 3-nitrobenzaldehyde, 4-nitrobenzaldehyde, 4-methyl-3-nitrobenzaldehyde, 3-hydroxy-4-nitrobenzaldehyde, 4-hydroxy-3-nitrobenzaldehyde, 5-hydroxy-2-nitrobenzaldehyde, 2-hydroxy-5-nitrobenzaldehyde, 2-hydroxy-3-nitrobenzaldehyde, 2-fluoro-3-nitrobenzaldehyde, 3-methoxy-2-nitrobenzaldehyde, 4-chloro-3-nitrobenzaldehyde, 2-chloro-6-nitrobenzaldehyde, 5-chloro-2-nitrobenzaldehyde, 4-chloro-2-nitrobenzaldehyde, 2,4-dinitrobenzaldehyde, 2,6-dinitrobenzaldehyde, 2-hydroxy-3-methoxy-5-nitrobenzaldehyde, 4,5-dimethoxy-2-nitrobenzaldehyde, 5-nitrovanillin, 3,5-dinitrosalicylaldehyde, 5-bromo-3-nitrosalicylaldehyde, 3-nitro-4-formylbenzenesulfonic acid, 4-nitro-1-naphthaldehyde, 2-nitrocinnamaldehyde, 3-nitrocinnamaldehyde, 4-nitrocinnamaldehyde, carbazole aldehydes or carbazole ketones, particularly 9-methyl-3-carbazolealdehyde, 9-ethyl-3-carbazolealdehyde, 3-acetylcarbazole, 3,6-diacetyl-9-ethylcarbazole, 3-acetyl-9-methylcarbazole, 1,4-dimethyl-3-carbazole aldehyde, 1,4,9-trimethyl-3-carbazolealdehyde, 4-trimethylammoniobenzaldehyde-, 4-benzyldimethylammoniobenzaldehyde-, 4-trimethylammoniocinnamaldehyde-, 4-trimethylammonionaphthaldehyde-, 2-methoxy-4-trimethylammoniobenzaldehyde-, N-(4-acetylphenyl)-trimethylammonium-, 4-(N,N-diethyl)-N-methylammonio)benzaldehyde-, N-(4-benzoylphenyl)trimethylammonium-, N-(4-benzoylphenyl)-N,N-diethylmethylammonium-, N-(4-formylphenyl)-N-methylpyrrolidinium-, N-(4-formylphenyl)-N-methylpiperidinium-, N-(4-formylphenyl)-N-methylmorpholinium-, N-(4-acetylphenyl)-N-methylmorpholinium-, N(4-benzoylphenyl)-N-methylmorpholinium-, 3-formyl-N-ethyl-N-methylcarbazolium-, 3-formyl-9,9-dimethylcarbazolium-, 1-(4-acetylphenyl)-3-methylimidazolium-, 1-(4-acetylphenyl)-3-methyl-2-imidazolinium-, 1-(4-benzoylphenyl)-3-methylimidazolium-, 5-acetyl-1,3-diethyl-2-methylbenzimidazolium-, 5-trimethylammonio-1-indanone-salts, particularly the benzenesulfonates, p-toluenesulfonates, methanesulfonates, ethanesulfonates, propanesulfonates, perchlorates, sulfates, chlorides, bromides, iodides, tetrachlorozincates, methylsulfates, trifluoromethanesulfonates, hexafluorophosphates, tetrafluoroborates,

4-formyl-1-methylpyridinium-, 2-formyl-1-methylpyridinium-, 4-formyl-1-ethylpyridinium-, 2-formyl-1-ethylpyridinium-, 4-formyl-1-benzylpyridinium-, 2-formyl-1-benzylpyridinium-, 4-formyl-1,2-dimethylpyridinium-, 4-formyl-1,3-dimethylpyridinium-, 4-formyl-1-methylquinolinium-, 2-formyl-1-methylquinolinium-, 4-acetyl-1-methylpyridinium-, 2-acetyl-1-methylpyridinium-, 4-acetyl-1-methylquinolinium-, 5-formyl-1-methylquinolinium-, 6-formyl-1-methylquinolinium-, 7-formyl-1-methylquinolinium-, 8-formyl-1-methylquinolinium, 5-formyl-1-ethylquinolinium-, 6-formyl-1-ethylquinolinium-, 7-formyl-1-ethylquinolinium-, 8-formyl-1-ethylquinolinium, 5-formyl-1-benzylquinolinium-, 6-formyl-1-benzylquinolinium-, 7-formyl-1-benzylquinolinium-, 8-formyl-1-benzylquinolinium, 5-formyl-1-allylquinolinium-, 6-formyl-1-allylquinolinium-, 7-formyl-1-allylquinolinium- and 8-formyl-1-allylquinolinium-, 5-acetyl-1-methylquinolinium-, 6-acetyl-1-methylquinolinium-, 7-acetyl-1-methylquinolinium-, 8-acetyl-1-methylquinolinium, 5-acetyl-1-ethylquinolinium-, 6-acetyl-1-ethylquinolinium-, 7-acetyl-1-ethylquinolinium-, 8-acetyl-1-ethylquinolinium, 5-acetyl-1-benzylquinolinium-, 6-acetyl-1-benzylquinolinium-, 7-acetyl-1-benzylquinolinium-, 8-acetyl-1-benzylquinolinium, 5-acetyl-1-allylquinolinium-, 6-acetyl-1-allylquinolinium-, 7-acetyl-1-allylquinolinium- and 8-acetyl-1-allylquinolinium, 9-formyl-10-methylacridinium-, 4-(2-formylvinyl)-1-methylpyridinium-, 1,3-dimethyl-2-(4-formylphenyl)-benzimidazolinium-, 1,3-dimethyl-2-(4-formylphenyl)-imidazolinium-, 2-(4-formylphenyl)-3-methylbenzothiazolium-, 2-(4-acetylphenyl)-3-methylbenzothiazolium-, 2-(4-formylphenyl)-3-methylbenzoxazolium-, 2-(5-formyl-2-furyl)-3-methylbenzothiazolium-, 2-(5-formyl-2-thienyl)-3-methylbenzothiazolium-, 2-(3-formylphenyl)-3-methylbenzothiazolium-, 2-(4-formylnaphth-1-yl)-3-methylbenzothiazolium-, 5-chloro-2-(4-formylphenyl)-3-methylbenzothiazolium-, 2-(4-formylphenyl)-3,5-dimethylbenzothiazolium-, 1-methyl-2-[2-(4-formylphenyl)-ethenyl]pyridinium-, 1-methyl-4-[2-(4-acetylphenyl)-ethenyl]pyridinium-, 1-benzyl-4-[2-(4-formylphenyl)-ethenyl]-pyridinium-, 1-methyl-4-[2-(4-formylphenyl)-ethenyl)pyridinium-, 1-methyl-2-[2-(4-formylphenyl)-ethenyl]pyridinium-, 1-methyl-4-[2-(4-formylphenyl)-ethenyl]quinolinium-, 1-methyl-2-[2-(4-formylphenyl)-ethenyl]-quinolinium-, 1-methyl-2-[2-(5-formyl-2-furyl)-ethenyl]-quinolinium-, 1-methyl-2-[2-(5-formyl-2-thienyl)-ethenyl]-quinolinium-, 1-methyl-2-[2-(4-formylphenyl)-ethenyl]benzothiazolinium-, 1,3-dimethyl-2-[2-(4-formylphenyl)-ethenyl]-benzimidazolinium-, 1,3-dimethyl-2-[2-(4-formylphenyl)-ethenyl]-imidazolinium-, 1-methyl-5-oxo-indeno[1,2-b]pyridinium(4-methyl-4-azonio-9-fluorenon-), 1-ethyl-5-oxo-indeno[1,2-b]pyridinium(4-ethyl-4-azonio-9-fluorenon-), 1-benzyl-5-oxoindeno[1,2-b]pyridinium(-4-benzyl-4-azonio-9-fluorenon-), 2-methyl-5-oxoindeno[1,2-c]pyridinium-, 2-methyl-9-oxo-indeno[2,1-c]pyridinium-, 1-methyl-9-oxoindeno[2,1-b]pyridinium-salts, particularly benzenesulfonate, p-toluenesulfonate, methanesulfonate, perchlorate, sulfate, chloride, bromide, iodide, tetrachlorozincate, methylsulfate, trifluoromethanesulfonate, tetrafluoroborate, salicylaldehyde, vanillin, 4-hydroxy-3-methoxycinnamaldehyde (coniferylaldehyde), 2,4-dihydroxybenzaldehyde, 4-dimethylaminobenzaldehyde, 4-diethylaminobenzaldehyde, 4-dimethylamino-2-hydroxybenzaldehyde, 4-pyrrolidinobenzaldehyde, 4-morpholinobenzaldehyde, 4-piperidinobenzaldehyde, 4-dimethylaminoacetophenone, 4-hydroxynaphthaldehyde, 4-dimethylaminonaphthaldehyde, 4-dimethylaminobenzylidenacetone, 4-dimethylaminocinnamaldehyde, 2-dimethylaminobenzaldehyde, 2-chloro-4-dimethylaminobenzaldehyde, 4-dimethylamino-2-methylbenzaldehyde, trans-4-diethylaminocinnamaldehyde, 4-(dibutylamino)benzaldehyde, 4-diphenylaminobenzaldehyde, 2,3,6,7-tetrahydro-1H,5H-benzo[ij]quinolizine-9-carboxaldehyde, 4-dimethylamino-2-methoxybenzaldehyde, 2,3,6,7-tetrahydro-8-hydroxy-1H,5H-benzo[ij]quinolizine-9-carboxaldehyde, 4-(1-imidazolyl)-benzaldehyde, 2-morpholinobenzaldehyde, indole-3-carboxaldehyde, 1-methylindole-3-carboxaldehyde, N-ethylcarbazole-3-carboxaldehyde, 2-formylmethylene-1,3,3-trimethylindoline (tribasic aldehyde) 1,3-diacetylbenzene, 1,4-diacetylbenzene, 1,3,5-triacetylbenzene, benzoyl-acetophenone, 2-(4′-methoxybenzoyl)acetophenone, 2-(2′-furoyl) acetophenone, 2-(2′-pyridoyl)acetophenone, 2-(3′-pyridoyl)acetophenone,

1-phenyl-1,2-propanedione, 1-phenyl-1,2-butanedione, 1-phenyl-3,3-dimethyl-1,2-butanedione, benzil, anisil, salicil, 5,5′-dibromosalicil, 2,2′-furil, 2,2′-thienil, 2,2′-, 4,4′-pyridil, 6,6′-dimethyl-4,4′-pyridil, 4-hydroxy-, 4-methoxy-, 4-chloro-, 4-methyl-, 4-dimethylamino-, 4,4′-dihydroxy-, -dimethyl-, -dibromo-, -dichloro-, -bisdimethylamino-, 2,4-dihydroxy-, 3,3′-dimethoxy, 2′-chloro-3,4-dimethoxy-, 3,4,5,3′,4′,5′-hexamethoxybenzil,

isatin derivatives, such as 5-chloroisatin, 5-methoxyisatin, 5-nitroisatin, 6-nitroisatin, 5-sulfoisatin, isatin-5-sulfonic acid, isatin-4-carboxylic acid and isatin-5-carboxylic acid,

N-substituted isatin derivatives, such as N-methylisatin, N-(2-hydroxyalkyl)-isatin, N-(2-hydroxypropyl)isatin, N-(3-hydroxypropyl)isatin, N-(2, 3-dihydroxypropyl) isatin, N-(2-sulfoethyl)isatin, (3-sulfopropyl)isatin, N-allylisatin, N-vinylisatin, N-benzylisatin, N-(4-methoxybenzyl)isatin, N-(4-carboxybenzyl)isatin, N-(4-sulfobenzyl)isatin, N-(2-dimethylaminoethyl)-isatin, N-(2-pyrrolidinoethyl)isatin, N-(2-piperidinoethyl)isatin, (2-morpholinoethyl)isatin, N-(2-furylmethyl)isatin, N-(thien-2-ylmethyl)isatin, N-(pyrid-2-ylmethyl)isatin, N-(pyrid-3-ylmethyl)isatin, N-(pyrid-4-ylmethyl)-isatin, N-allylisatin-5-sulfonic acid, 5-chloro-N-(2-hydroxyethyl)isatin, 5-methyl-N-(2-hydroxyethyl)isatin, 5,7-dichloro-N-allylisatin, 5-nitro-N-allylisatin, N-hydroxymethylisatin, N-hydroxymethyl-5-methylisatin, N-hydroxymethyl-5-chloroisatin, N-hydroxymethyl-5-sulfoisatin, N-hydroxymethyl-5-carboxyisatin, N-hydroxymethyl-5-nitroisatin, N-hydroxymethyl-5-bromoisatin, N-hydroxymethyl-5-methoxyisatin, N-hydroxymethyl-5,7-dichloroisatin, N-dimethylaminomethylisatin, N-diethylaminomethylisatin, N-(bis(2-hydroxyethyl)aminomethyl)isatin, N-(2-hydroxyethylaminomethyl)isatin, N-(bis-(2-hydroxypropyl)aminomethyl)-isatin, N-pyrrolidinomethylisatin, N-piperidinomethylisatin, N-morpholinomethylisatin, N-(1,2,4-triazol-1-ylmethyl)isatin, N-(imidazol-1-ylmethyl)isatin, N-carboxymethylaminomethylisatin, N-(2-carboxyethylaminomethyl)isatin, N-(3-carboxypropylaminomethyl)isatin, N-(bis(2-hydroxyethyl)aminomethyl)-5-methylisatin, N-piperidinomethyl-5-chloroisatin, N-(2-sulfoethylamino)isatin, as well as the alkali- and optionally ammonium salts of the acidic compounds, quinisatin and their derivatives, such as N-methylquinisatin,

acetophenone, propiophenone, 2-hydroxyacetophenone, 3-hydroxyacetophenone, 4-hydroxyacetophenone, 2-hydroxypropiophenone, 3-hydroxypropiophenone, 4-hydroxypropiophenone, 2-hydroxybutyrophenone, 3-hydroxybutyrophenone, 4-hydroxybutyrophenone, 2,4-dihydroxyacetophenone, 2,5-dihydroxyacetophenone, 2,6-dihydroxyacetophenone, 2,3,4-trihydroxyacetophenone, 3,4,5-trihydroxyacetophenone, 2,4,6-trihydroxyacetophenone, 2,4,6-trimethoxyacetophenone, 3,4,5-trimethoxyacetophenone, 3,4,5-trimethoxy-acetophenone-diethylketal, 4-hydroxy-3-methoxy-acetophenone, 3,5-dimethoxy-4-hydroxy-acetophenone, 4-amino-acetophenone, 4-dimethylamino-acetophenone, 4-morpholino-acetophenone, 4-piperidinoacetophenone, 4-imidazolino-acetophenone, 2-hydroxy-5-bromo-acetophenone, 4-hydroxy-3-nitroacetophenone, acetophenone-2-carboxylic acid, acetophenone-4-carboxylic acid, benzophenone, 4-hydroxybenzophenone, 2-amino-benzophenone, 4,4′-dihydroxybenzophenone, 2,4-dihydroxybenzophenone, 2,4,4′-trihydroxybenzophenone, 2,3,4-trihydroxybenzophenone, 2-hydroxy-1-acetonaphthone, 1-hydroxy-2-acetonaphthone, chromone, chromone-2-carboxyic acid, flavone, 3-hydroxyflavone, 3,5,7-trihydroxyflavone, 4′,5,7-trihydroxyflavone, 5,6,7-trihydroxyflavone, quercetin, indanone, 9-fluorenone, 3-hydroxyfluorenone, anthrone, 1,8-dihydroxyanthrone,

heterocyclic carbonyl compounds, such as 2-indolaldehyde, 3-indolaldehyde, 1-methylindol-3-aldehyde, 2-methylindol-3-aldehyde, 1-acetylindol-3-aldehyde, 3-acetylindole, 1-methyl-3-acetylindole, 2-(1,3,3-trimethyl-2-indolinylidene)acetaldehyde, 1-methylpyrrol-2-aldehyde, 1-methyl-2-acetylpyrrole, 1-pyridinealdehyde, 2-pyridinealdehyde, 3-pyridinealdehyde, 4-acetylpyridine, 2-acetylpyridine, 3-acetylpyridine, pyridoxal, quinoline-3-aldehyde, quinoline-4-aldehyde, antipyrine-4-aldehyde, furfural, 5-nitrofurfural, 2-thenoyl-trifluoroacetone, chromone-3-aldehyde, 3-(5-nitro-2-furyl)acrolein, 3-(2-furyl)acrolein, imidazole-2-aldehyde, 1,3-diimino-isoindoline,

indanone derivatives, such as, e.g., 1,2-indandione, 2-oximo-1-indanone, indan-1,2,3-trione-2-oxime, 5-methoxy-indan-1,2,3-trione-2-oxime, 2- nitro-1,3-indandione

as well as physiologically acceptable salts of the abovementioned compounds.

The reactive carbonyl compounds are preferably each used in an amount of 0.03 to 65 mmol, particularly 1 to 40 mmol, based on 100 g of the added finished dyeing agent.

The added compounds with the Formulae C1-C22 and I, IV, V, Vla and Vlb are in the main known in the literature or are commercially available or can be synthesized using known synthetic processes.

Colorations with an even higher brilliance and improved fastness properties (light fast, wash fast, rub fast) over a broad spectrum of shades are obtained if the agent according to the invention comprises at least an additional component (hereafter named component D), selected from (a) compounds with primary or secondary amino groups or hydroxyl groups, selected from primary or secondary aromatic amines, nitrogen-containing heterocyclic compounds and aromatic hydroxy compounds, (b) amino acids, (c) oligopeptides constructed from 2 to 9 amino acids and their physiologically acceptable salts. The compounds of component D are on the one hand, compounds that by themselves, only weakly dye keratin-containing fibers and for example first produce brilliant colorations together with the component C. On the other hand, however, there are also compounds that are already added as oxidation dye precursors.

The primary and secondary aromatic amines of component D are preferably selected from the group consisting of N,N-dimethyl-p-phenylenediamine, N,N-diethyl-p-phenylenediamine, N-(2-hydroxyethyl)-N-ethyl-p-phenylenediamine, N,N-bis-(2-hydroxyethyl)-p-phenylenediamine, N-(2-methoxyethyl)-p-phenylenediamine, 2,3-dichloro-p-phenylenediamine, 2,4-dichloro-p-phenylenediamine, 2,5-dichloro-p-phenylenediamine, 2-chloro-p-phenylenediamine, 2,5-dihydroxy-4-morpholinoaniline, 2-aminophenol, 3-aminophenol, 4-aminophenol, 2-aminomethyl-4-aminophenol, 2-hydroxymethyl-4-aminophenol, o-phenylenediamine, m-phenylenediamine, p-phenylenediamine, 2,5-diaminotoluene, 2,5-diaminophenol, 2,5-diaminoanisole, 2,5-diaminophenethol, 4-amino-3-methylphenol, 2-(2,5-diaminophenyl)ethanol, 2,4-diaminophenoxyethanol, 2-(2,5-diaminophenoxy)ethanol, 3-amino-4-(2-hydroxyethyloxy)phenol, 3,4-methylenedioxyphenol, 3,4-methylenedioxyaniline, 3-amino-2,4-dichlorophenol, 4-methylaminophenol, 2-methyl-5-aminophenol, 3-methyl-4-aminophenol, 2-methyl-5-(2-hydroxyethylamino)phenol, 3-amino-2-chloro-6-methylphenol, 2-methyl-5-amino-4-chlorophenol, 5-(2-hydroxyethylamino)-4-methoxy-2-methylphenol, 4-amino-2-hydroxymethylphenol, 2-(diethylaminomethyl)-4-aminophenol, 4-amino-1-hydroxy-2-(2-hydroxyethylaminomethyl)benzene, 1-hydroxy-2-amino-5-methyl-benzene, 1-hydroxy-2-amino-6-methylbenzene, 2-amino-5-acetamidophenol, 1,3-dimethyl-2,5-diaminobenzene, 5-(3-hydroxypropylamino)-2-methylphenol, 5-amino-4-methoxy-2-methylphenol, N,N-dimethyl-3-aminophenol, N-cyclopentyl-3-aminophenol, 5-amino-4-fluoro-2-methylphenol, 2,4-diamino-5-fluorotoluene, 2,4-diamino-5-(2-hydroxyethoxy)-toluene, 2,4-diamino-5-methylphenetol, 3,5-diamino-2-methoxy-1-methylbenzene, 2-amino-4-(2-hydroxyethylamino)anisole, 2,6-bis-(2-hydroxyethylamino)-1-methylbenzene, 1,3-diamino-2,4-dimethoxybenzene, 3,5-diamino-2-methoxy-toluene, 2-aminobenzoic acid, 3-aminobenzoic acid, 4-aminobenzoic acid, 2-aminophenylacetic acid, 3-aminophenylacetic acid, 4-aminophenylacetic acid, 2,3-diaminobenzoic acid, 2,4-diaminobenzoic acid, 2,5-diaminobenzoic acid, 3,4-diaminobenzoic acid, 3,5-diaminobenzoic acid, 4-aminosalicylic acid, 5-aminosalicylic acid, 3-amino-4-hydroxybenzoic acid, 4-amino-3-hydroxybenzoic acid, 2-aminobenzenesulfonic acid, 3-aminobenzenesulfonic acid, 4-aminobenzenesulfonic acid, 3-amino-4-hydroxybenzenesulfonic acid, 4-amino-3-hydroxynaphthalene-1-sulfonic acid, 6-amino-7-hydroxynaphthalene-2-sulfonic acid, 7-amino-4-hydroxynaphthalene-2-sulfonic acid, 4-amino-5-hydroxynaphthalene-2,7-disulfonic acid, 3-amino-2-naphthoic acid, 3-aminophthalic acid, 5-aminoisophthalic acid, 1,3,5-triaminobenzene, 1,2,4-triaminobenzene, 1,2,4,5-tetraaminobenzene, 2,4,5-triaminophenol, pentaaminobenzene, hexaaminobenzene, 2,4,6-triaminoresorcine, 4,5-diaminopyrocatechol, 4,6-diaminopyrogallol, 1-(2-hydroxy-5-aminobenzyl)-2-imidazolidinone, 4-amino-2((4-[(5-amino-2-hydroxyphenyl)methyl]-piperazinyl)methyl)phenol, 3,5-diamino-4-hydroxypyrocatechol, 1,4-bis-(4-aminophenyl)-1,4-diazacycloheptane, aromatic nitriles, such as 2-amino-4-hydroxybenzonitrile, 4-amino-2-hydroxybenzonitrile, 4-aminobenzonitrile, 2,4-diaminobenzonitrile, amino compounds with nitro groups, such as 3-amino-6-methylamino-2-nitropyridine, picramic acid, [8-[(4-amino-2-nitrophenyl)-azo]-7-hydroxy-naphth-2-yl]trimethylammonium chloride, [8-[(4-amino-3-nitrophenyl)-azo)-7-hydroxy-naphth-2-yl]trimethylammonium chloride (Basic Brown 17), 1-hydroxy-2-amino-4,6-dinitrobenzene, 1-amino-2-nitro-4-[bis(2-hydroxyethyl)amino]benzene, 1-amino-2-[(2-hydroxyethyl)amino]-5-nitrobenzene (HC Yellow Nr. 5), 1-amino-2-nitro-4-[(2-hydroxyethyl)amino]benzene (HC Red Nr. 7), 2-chloro-5-nitro-N-2-hydroxyethyl-1,4-phenylenediamine, 1-[(2-hydroxyethyl)amino]-2-nitro-4-aminobenzene (HC Red Nr. 3), 4-amino-3-nitrophenol, 4-amino-2-nitrophenol, 6-nitro-o-toluidine, 1-amino-3-methyl-4-[(2-hydroxyethyl)amino]-6-nitrobenzene (HC Violet Nr. 1), 1-amino-2-nitro-4-[(2,3-dihydroxypropyl)amino]-5-chlorobenzene (HC Red Nr. 10), 2-(4-amino-2-nitroanilino)benzoic acid, 6-nitro-2,5-diaminopyridine, 2-amino-6-chloro-4-nitrophenol, disodium salt of 1-amino-2-(3-nitrophenylazo)-7-phenylazo-8-naphthol-3,6-disulfonic acid (acid blue Nr.29), disodium salt of 1-amino-2-(2-hydroxy-4-nitrophenylazo)-8-naphthol-3,6-disulfonic acid (Palatinchrome green), disodium salt of 1-amino-2-(3-chloro-2-hydroxy-5-nitrophenylazo)-8-naphthol-3,6-disulfonic acid (Gallion), disodium salt of 4-amino-4′-nitrostilbene-2,2′-disulfonic acid, 2,4-diamino-3′,5′-dinitro-2′-hydroxy-5-methylazobenzene (Mordant brown 4), 4′-amino-4-nitrodiphenylamine-2-sulfonic acid, 4′-amino-3′-nitrobenzophenone-2-carboxylic acid, 1-amino-4-nitro-2-(2-nitrobenzylideneamino)benzene, 2-[2-(diethylamino)ethylamino]-5-nitroaniline, 3-amino-4-hydroxy-5-nitrobenzenesulfonic acid, 3-amino-3′-nitrobiphenyl, 3-amino-4-nitroacenaphthene, 2-amino-1-nitronaphthalene, 5-amino-6-nitrobenzo-1,3-dioxol, anilines, particularly anilines containing nitro groups, such as 4-nitroaniline, 2-nitroaniline, 1,4-diamino-2-nitrobenzene, 1,2-diamino-4-nitrobenzene, 1-amino-2-methyl-6-nitrobenzene, 4-nitro-1,3-phenylenediamine, 2-nitro-4-amino-1-(2-hydroxyethylamino)benzene, 2-nitro-1-amino-4-[bis(2-hydroxyethyl)amino]benzene, 4-amino-2-nitrodiphenylamine-2′-carboxylic acid, 1-amino-5-chloro-4-(2-hydroxyethylamino)-2-nitrobenzene, aromatic anilines or phenols with a further aromatic radical, as illustrated in Formula VII 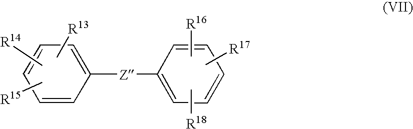
in which
R¹³ stands for a hydroxyl or an amino group, which can be substituted with a C₁-C₆ alkyl group, a C₁-C₆ hydroxyalkyl group, a C₁-C₆ alkoxy group or a C₁-C₆ alkoxy C₁-C₆ alkyl group,
R¹⁴, R¹⁵, R¹⁶, R¹⁷ and R¹⁸ independently of one another stand for a hydrogen atom, a hydroxyl or an amino group, which can be substituted with a C₁-C₆ alkyl group, a C₁-C₆ hydroxyalkyl group, a C₁-C₆ alkoxy group, a C₁-C₆ aminoalkyl group or a C₁-C₆ alkoxy C₁-C₆ alkyl group, and
Z″ is a direct bond, a saturated or unsaturated carbon chain with 1 to 4 carbon atoms, optionally substituted with hydroxyl groups, a carbonyl-, sulfonyl- or imino group, an oxygen- or sulfur atom, or a group with the Formula VIII,
   —Q′ [symbol] CH₂—Q—CH₂—Q″ [symbol] ₒ VIII)
   in which
Q means a direct bond, a CH₂— or CHOH group,
Q′ and Q″ independently of one another stand for an oxygen atom, an NR¹⁹ group, in which R¹⁹ means a hydrogen atom, a C₁-C₆ alkyl group or C₁-C₆ hydroxyalkyl group, wherein also both groups, together with the remainder of the molecule, can form a 5-, 6- or 7-membered ring, the groups O—(CH₂)ₚ—NH or NH—(CH₂)_{p′}—O, in which p and p′ are 2 or 3, and
o means a number from 1 to 4,
   such as, for example 4,4′-diaminostilbene and its hydrochloride, mono- or di-Na-salt of 4,4′-diaminostilbene-2,2′-disulfonic acid, 4-amino-4′-dimethylaminostilbene and its hydrochloride, 4,4′-diaminodiphenylmethane, 4,4′-diaminodiphenylsulfide, 4,4′-diaminodiphenylsulfoxide, 4,4′-diaminodiphenylamine, 4,4′-diaminodiphenylamine-2-sulfonic acid, 4,4′-diaminobenzophenone, 4,4′-diaminodiphenylether, 3,3′,4,4′-tetraaminodiphenyl, 3,3′,4,4′-tetraaminobenzophenone, 1,3-bis-(2,4-diaminophenoxy)propane, 1,8-bis-(2,5-diaminophenoxy)-3,6-dioxaoctane, 1,3-bis-(4-aminophenylamino)propane, 1,3-bis-(4-aminophenylamino)-2-propanol, 1,3-bis-[N-(4-aminophenyl)-2-hydroxyethylamino]-2-propanol, N,N-bis-[2-(4-aminophenoxy)ethyl]methyl amine, N-phenyl-1,4-phenylenediamine and bis(5-amino-2-hydroxyphenyl)methane.

The nitrogen-containing heterocyclic compounds of component D are preferably selected from the group consisting of 2-aminopyridine, 3-aminopyridine, 4-aminopyridine, 2-amino-3-hydroxypyridine, 2,6-diaminopyridine, 2,5-diaminopyridine, 2-(aminoethylamino)-5-aminopyridine, 2,3-diaminopyridine, 2-dimethylamino-5-aminopyridine, 2-methylamino-3-amino-6-methoxypyridine, 2,3-diamino-6-methoxypyridine, 2,6-dimethoxy-3,5-diaminopyridine, 2,4,5-triaminopyridine, 2,6-dihydroxy-3,4-dimethylpyridine, N-[2-(2,4-diaminophenyl)aminoethyl]-N-(5-amino-2-pyridyl)amine, N-[2-(4-aminophenyl)aminoethyl]-N-(5-amino-2-pyridyl)amine, 2,4-dihydroxy-5,6-diaminopyrimidine, 4,5,6-triaminopyrimidine, 4-hydroxy-2,5,6-triaminopyrimidine, 2-hydroxy-4,5,6-triaminopyrimidine, 2,4,5,6-tetraaminopyrimidine, 2-methylamino-4,5,6-triaminopyrimidine, 2,4-diaminopyrimidine, 4,5-diaminopyrimidine, 2-amino-4-methoxy-6-methyl-pyrimidine, 3,5-diaminopyrazole, 3,5-diamino-1,2,4-triazole, 3-aminopyrazole, 3-amino-5-hydroxypyrazole, 1-phenyl-4,5-diaminopyrazole, 1-(2-hydroxyethyl)-4,5-diaminopyrazole, 1-phenyl-3-methyl-4,5-diaminopyrazole, 4-amino-2,3-dimethyl-1-phenyl-3-pyrazolin-5-one (4-aminoantipyrine), 1-phenyl-3-methylpyrazol-5-one, 2-aminoquinoline, 3-aminoquinoline, 8-aminoquinoline, 4-aminoquinaldine, 2-aminonicotinic acid, 6-aminonicotinic acid, 5-aminoisoquinoline, 5-aminoindazole, 6-aminoindazole, 5-aminobenzimidazole, 7-aminobenzimidazole, 5-aminobenzothiazole, 7-aminobenzothiazole, 2,5-dihydroxy-4-morpholino-aniline as well as indole- and indoline derivates, such as 4-aminoindole, 5-aminoindole, 6-aminoindole, 7-aminoindole, 5,6-dihydroxyindole, 5,6-dihydroxyindoline, 4-hydroxyindoline. In addition, the heterocyclic compounds 4-hydroxypyrimidines, disclosed in DE-U1-299 08 573, may be added according to the invention. The above compounds may be added both in their free form as well as in the form of their physiologically acceptable salts, e.g., salts of inorganic acids, such as hydrochloric acid or sulfuric acid.

The aromatic hydroxyl compounds of component D are preferably selected from the group consisting of 2-, 4-, 5-methylresorcinol, 2,5-dimethylresorcinol, resorcinol, 3-methoxyphenol, pyrocatechol, hydroquinone, pyrogallol, phloroglucinol, hydroxyhydroquinone, 2-, 3-, 4-methoxy-, 3-dimethylamino-, 2-(2-hydroxyethyl)-, 3,4-methylenedioxyphenol, 2,4-, 3,4-dihydroxybenzoic acid, -phenylacetic acid, gallic acid, 2,4,6-trihydroxybenzoic acid, -acetophenone, 2-, 4-chlororesorcinol, 1-naphthol, 1,5-, 2,3-, 2,7-dihydroxynaphthalene, 6-dimethylamino-4-hydroxy-2-naphthalenesulfonic acid and 3,6-dihydroxy-2,7-naphthalenesulfonic acid.

Preferred amino acids include all naturally occurring and synthetic α-amino acids, e.g. those amino acids obtained by hydrolysis of vegetable or animal proteins, e.g., collagen, keratin, casein, elastin, soy protein, wheat gluten or almond protein. In this respect, both acidic and alkaline reactive amino acids may be used. Preferred amino acids are arginine, histidine, tyrosine, phenylalanine, DOPA (dihydroxyphenylalanine), ornithine, proline, lysine and tryptophan. However, other amino acids, such as 6-aminocaproic acid and β-alanine may also be used.

Again, the oligopeptides can be naturally occurring or synthetic oligopeptides, and also those contained in polypeptide hydrolyzates or protein hydrolysates, in so far as they are sufficiently water-soluble for use in the process according to the invention. Examples of oligoproteins are e.g. glutathione or those contained in the hydrolyzates of collagen, keratin, casein, elastin, soy protein, wheat gluten or almond protein. Here, their use is preferred together with compounds containing primary or secondary amino groups or with aromatic hydroxyl compounds.

Particularly preferred compounds of component D are selected from the group consisting of N-(2-hydroxyethyl)-N-ethyl-p-phenylenediamine, 2-chloro-p-phenylenediamine, N,N-bis-(2-hydroxyethyl)-p-phenylenediamine, 2-aminophenol, 3-aminophenol, 4-aminophenol, 2-amino-6-chloro-4-nitrophenol, p-phenylenediamine, 2-(2,5-diaminophenyl)ethanol, 2,5-diaminotoluene, 3,4-methylenedioxyaniline, 2-amino-4-(2-hydroxyethylamino)anisole, 2-(2,4-diaminophenoxy)ethanol, 3-amino-2,4-dichlorophenol, 2-methyl-5-aminophenol, 3-methyl-4-aminophenol, 2-methyl-5-(2-hydroxyethylamino)phenol, 2-methyl-5-amino-4-chlorophenol, 6-methyl-3-amino-2-chlorophenol, 2-aminomethyl-4-aminophenol, 2-diethylaminomethyl-4-aminophenol, 2-dimethylaminomethyl-4-aminophenol, 2,6-dichloro-4-aminophenol, 2-hydroxymethyl-4-aminophenol, 2,6-bis(2-hydroxyethylamino)-1-methylbenzene, bis(2-hydroxy-5-aminophenyl)methane, bis-(4,5-amino-2-hydroxyphenyl)methane, 1,3-bis(2,4-diaminophenoxy)propane, 1,4-bis(4-aminophenyl)-1,4-diazacycloheptane, 1,8-bis(2,5-diaminophenoxy)-3,6-dioxaoctane, 4,4′-diaminodiphenylamine, 3,4-methylenedioxyphenol, 3,4-diaminobenzoic acid, 2,5-diaminopyridine, 2-dimethylamino-5-aminopyridine, 2-amino-3-hydroxypyridine, 3-amino-2-methylamino-6-methoxypyridine, 2,3-diamino-6-methoxypyridine, 3,5-diamino-2,6-dimethoxypyridine, 2,6-dihydroxy-3,4-dimethylpyridine, 2-hydroxy-4,5,6-triaminopyrimidine, 4-hydroxy-2,5,6-triaminopyrimidine, 2,4,5,6-tetraaminopyrimidine, 2-methylamino-4,5,6-triamino-pyrimidine, 3,5-diaminopyrazole, 3-amino-5-hydroxypyrazole, 4,5-diamino-1-(2-hydroxyethyl)pyrazole, 5,6-dihydroxyindole, 5,6-dihydroxyindoline, 4-amino-2,3-dimethyl-1-phenyl-3-pyrazolin-5-one (4-aminoantipyrine), β-alanine, L-proline, L-lysine, DL-tyrosine as well as their physiologically acceptable salts of preferably inorganic acids.

Quite particularly preferred compounds of component D according to the invention, are N-(2-hydroxyethyl)-N-ethyl-p-phenylenediamine, 2,5-diaminotoluene, 2-(2,5-diaminophenyl)ethanol, 2-aminophenol, 3-aminophenol, 4-aminophenol, 2-aminomethyl-4-aminophenol, 2-(diethylaminomethyl)-4-aminophenol, 1-hydroxy-2-amino-5-methylbenzene, 2,4-diaminophenoxyethanol, 3-amino-2,4-dichlorophenol, 2-methyl-5-aminophenol, 2-methyl-5-(2-hydroxyethylamino)phenol, 6-methyl-3-amino-2-chlorophenol, 2-methyl-5-amino-4-chlorophenol, 3,4-methylenedioxyaniline, 3,4-methylenedioxyaniline, 3,4-diaminobenzoic acid, 3,5-diamino-2-methoxy-1-methylbenzene, 3,5-diamino-2-methoxy-1-methylbenzene, 2-amino-4-(2-hydroxyethylamino)anisole, 2,6-bis(2-hydroxyethylamino)-1-methylbenzene, 1,3-bis(2,4-diaminophenoxy)propane, 4,5-diamino-1-(2-hydroxyethyl)pyrazole, 1,4-bis(4-aminophenyl)-1,4-diazacycloheptane, bis-(2-hydroxy-5-aminophenyl)methane, 1,8-bis-(2,5-diaminophenoxy)-3,6-dioxaoctane, 4,4′-diaminodiphenylamine, 2-amino-3-hydroxypyridine, 3-amino-2-methylamino-6-methoxypyridine, 2,6-dihydroxy-3,4-dimethylpyridine, 5,6-dihydroxyindole, 5,6-dihydroxyindoline, 2,4,5,6-tetraaminopyrimidine, 2-hydroxy-4,5,6-triamino-pyrimidine, 4-hydroxy-2,5,6-triaminopyrimidine as well as their physiologically acceptable salts of preferably inorganic acids.

The abovementioned compounds of component D can each be added in an amount of 0.03 to 65 mmol, particularly 1 to 40 mmol, based on 100 g of the added finished dyeing agent.

In the process according to the invention, additional and more intensive colorations can be achieved by adding additional color enhancers. Preferably, the color enhancers are selected from the group consisting of piperidine, piperidine-2-carboxylic acid, piperidine-3-carboxylic acid, piperidine-4-carboxylic acid, pyridine, 2-hydroxypyridine, 3-hydroxypyridine, 4-hydroxypyridine, imidazole, 1-methylimidazole, histidine, pyrrolidine, proline, pyrrolidone, pyrrolidone-5-carboxylic acid, pyrazole, 1,2,4-triazole, piperazine, methoxybutanol, propylene carbonate, ethylene carbonate, their derivatives as well as their physiologically acceptable salts.

The above-mentioned color enhancers can each be added in an amount of 0.03 to 65 mmol, particularly 1 to 40 mmol, based on 100 g of the total dyeing agent.

In a further embodiment for further modification of the color shades, additional customary substantive dyes, e.g., from the group of nitrophenylenediamine, nitroaminophenols, anthraquinone or indophenols, such as, e.g., the known compounds under the international designations or trade names HC Yellow 2, HC Yellow 4, HC Yellow 6, Basic Yellow 57, Disperse Orange 3, HC Red BN, Basic Red 76, HC Blue 2, Disperse Blue 3, Basic Blue 99, HC Violet 1, Disperse Violet 1, Disperse Violet 4, Disperse Black 9, Basic Brown 16 and Basic Brown 17, as well as 6-nitro-1,2,3,4-tetrahydroquinoxaline, 4-N-ethyl-1,4-bis(2′-hydroxyethylamino)-2-nitrobenzene hydrochloride and 1-methyl-3-nitro-4-(2′-hydroxyethylamino)benzene are added to the agent according to the invention in addition to the components used according to the invention. According to this embodiment, in the process according to the invention, the substantive dyes are preferably added in an amount of 0.01 to 20 wt. % based on the added components.

In addition, the preparations used according to the invention can also comprise naturally occurring dyes, such as henna red, henna neutral, henna black, camomille leaves, sandalwood, black tea, alder bark, sage, logwood, madder root, cashew, cedar and alkanet root.

Concerning additional customary dye components, reference is expressly made to the series “Dermatology”, published by Ch. Culnan, H. Maibach, Verlag Marcel Dekker Inc., New York, Basel, 1986, Vol. 7, Ch. Zviak, The Science of Hair Care, ch. 7, pages 248-250 (substantive dyes), and ch. 8, pages 264-267 (oxidation dyes), as well as the “Europäische Inventar der Kosmetikrohstoffe”, 1996, published by the European Commission, available in a disk format from the Bundesverband der deutschen Industrie- und Handelsunternehmen für Arzneimittel, Reformwaren und Körperpflegemittel e. V., Mannheim.

Each of the compounds comprised according to the invention with the Formulae C1-C22, I, IV, V, Vla and Vlb or the optionally comprised color enhancers and substantive dyes are not required to be pure compounds. In fact, the dyeing agents used according to the invention can comprise minor amounts of additional components resulting from the manufacturing processes for the individual dyes, in so far as these do not detrimentally influence the coloration result or must be excluded on other grounds, e.g., toxicological.

The dyeing agents used according to the invention already produce intensive colorations at physiologically acceptable temperatures below 45° C. They are therefore particularly suited for dyeing human hair. The dyeing agents are normally mixed with an aqueous cosmetic carrier for application on human hair. Suitable aqueous cosmetic carriers are e.g. creams, emulsions, gels or also foaming solutions that contain surfactants, such as e.g. shampoos or other preparations that are suitable for application on keratin-containing fibers. If required, it is also possible to mix the dyeing agent with anhydrous carriers.

Furthermore, the dyeing agents used according to the invention can comprise all known active ingredients, additives and auxiliaries for such preparations. In many cases, the dyeing agents comprise at least a surfactant, both anionic and also zwitterionic, ampholytic, nonionic and cationic surfactants being suitable in principle. In many cases, however, it has proved to be advantageous to select the surfactant from anionic, zwitterionic or nonionic surfactants.

Suitable anionic surfactants for the preparations according to the invention are any anionic surface-active substances suitable for use on the human body. Such substances are characterized by a water-solubilizing anionic group such as, for example, a carboxylate, sulfate, sulfonate or phosphate group and a lipophilic alkyl group containing around 10 to 22 carbon atoms. In addition, glycol or polyglycol ether groups, ester, ether, amide and hydroxyl groups may also be present in the molecule. The following are examples of suitable anionic surfactants, each in the form of the sodium, potassium and ammonium salts as well as mono-, di- and trialkanolammonium salts containing 2 or 3 carbon atoms in the alkanol group,

linear fatty acids containing 10 to 22 carbon atoms (soaps),
ether carboxylic acids with the formula R—O—(CH₂—CH₂O)ₓ—CH₂—COOH, in which R is a linear alkyl group containing 10 to 22 carbon atoms and x=0 or 1 to 16,
acyl sarcosides containing 10 to 18 carbon atoms in the acyl group,
acyl taurides containing 10 to 18 carbon atoms in the acyl group,
acyl isethionates containing 10 to 18 carbon atoms in the acyl group,
mono- and dialkyl sulfosuccinates containing 8 to 18 carbon atoms in the alkyl group and monoalkyl polyoxyethyl sulfosuccinates containing 8 to 18 carbon atoms in the alkyl group and 1 to 6 oxyethyl groups,
linear alkanesulfonates containing 12 to 18 carbon atoms,
linear alpha-olefinesulfonates containing 12 to 18 carbon atoms,
methyl esters of alpha-sulfofatty acids from fatty acids containing 12 to 18 carbon atoms,
alkyl sulfates and alkyl polyglycol ether sulfates corresponding to the formula R—O(CH₂—CH₂O)ₓ—OSO₃H, in which R is a preferably linear alkyl group containing 10 to 18 carbon atoms and x=0 or 1 to 12,
mixtures of surface-active hydroxysulfonates according to DE-A-37 25 030,
sulfated hydroxyalkylpolyethylene- and/or hydroxyalkylenepropylene glycol ethers according to DE-A-37 23 354,
sulfonates of unsaturated fatty acids containing 12 to 24 carbon atoms and 1 to 6 double bonds according to DE-A-39 26 344,
esters of tartaric acid and citric acid with alcohols in the form of addition products of around 2 to 15 molecules of ethylene oxide and/or propylene oxide with fatty alcohols containing 8 to 22 carbon atoms.

Preferred anionic surfactants are alkyl sulfates, alkyl polyglycol ether sulfates and ether carboxylic acids containing 10 to 18 carbon atoms in the alkyl group and up to 12 glycol ether groups in the molecule as well as particularly salts of saturated and particularly unsaturated C₈-C₂₂ carboxylic acids such as oleic acid stearic acid, isostearic acid and palmitic acid.

Zwitterionic surfactants are defined as surface-active compounds, which contain at least one quaternary ammonium group and at least one —COO⁽⁻⁾ or —SO₃⁽⁻⁾ group in the molecule. Particularly suitable zwitterionic surfactants are the so-called betaines, such as N-alkyl-N,N-dimethylammonium glycinates, for example cocoalkyldimethylammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. A preferred zwitterionic surfactant is the fatty acid amide derivative known by the CTFA name of Cocamidopropyl Betaine.

Ampholytic surfactants are understood to be such surface-active compounds which, in addition to a C₈-C₁₈ alkyl or acyl group, contain at least one free amino group and at least one —COOH or —SO₃H group in the molecule and which are capable of forming internal salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids each containing around 8 to 18 carbon atoms in the alkyl group. Particularly preferred ampholytic surfactants are N-cocoalkyl aminopropionate, cocoacyl aminoethyl aminopropionate and C₁₂-C₁₈ acylsarcosine.

Nonionic surfactants contain as the hydrophilic group, for example a polyol group, a polyalkylene glycol ether group or a combination of polyol and polyglycol ether groups. Examples of such compounds are
products of the addition of 2 to 30 mol ethylene oxide and/or 0 to 5 mol propylene oxide onto linear and branched fatty alcohols containing 8 to 22 carbon atoms, onto fatty acids containing 12 to 22 carbon atoms and onto alkylphenols containing 8 to 15 carbon atoms in the alkyl group,
C₁₂-C₂₂ fatty acid monoesters and diesters of products of the addition of 1 to 30 mol ethylene oxide onto glycerol,
C₈-C₂₂ alkylmono- and oligoglycosides and their ethoxylated analogs, products of the addition of 5 to 60 mol ethylene oxide onto castor oil and hydrogenated castor oil,
products of the addition of ethylene oxide onto sorbitan fatty acid esters,
products of the addition of ethylene oxide onto fatty acid alkanolamides.

Examples of cationic surfactants suitable for the agents used according to the invention are particularly quaternary ammonium compounds. Ammonium halides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride. Other suitable cationic surfactants according to the invention are represented by quaternized protein hydrolyzates.

According to the invention, also suitable are cationic silicone oils such as, for example the commercially available products Q2-7224 (manufacturer: Dow Corning; a stabilized trimethylsilylamodimethicone), Dow Corning® 929 Emulsion (comprising a hydroxylamino-modified silicone, which is also referred to as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxanes, Quaternium-80).

Alkylamidoamines, particularly fatty acid amidoamines such as stearylamido propyl dimethyl amine, available under the name Tego Amid®S 18, are characterized by a good conditioning action, especially by their good biodegradability.

Quaternary ester compounds, known as “esterquats”, such as methylhydroxyalkyldialkoyloxyalkylammonium methosulfate commercialized under the trade name Stepantex®, likewise have good biodegradability.

An example of a suitable cationic surfactant quaternary sugar derivative is the commercial product Glucquat®100, a “lauryl methyl gluceth-10 hydroxypropyl dimonium chloride” according to CTFA nomenclature.

The compounds used as surfactants with alkyl groups may each be homogeneous compounds. In general, however, these compounds are preferably produced from natural vegetal or animal raw materials and result in mixtures of products with raw material-dependent, differing alkyl chain lengths.

The surfactants representing addition products of ethylene and/or propylene oxide with fatty alcohols or derivatives of these addition products may be both products with a “normal” homolog distribution and products with a narrow homolog distribution. Products with a “normal” homolog distribution are mixtures of homologs which are obtained from the reaction of fatty alcohol and alkylene oxide using alkali metals, alkali metal hydroxides or alkali metal alcoholates as catalysts. By contrast, narrow homolog distributions are obtained when, for example, hydrotalcites, alkaline earth metal salts of ether carboxylic acids, alkaline earth metal oxides, hydroxides or alcoholates are used as catalysts. The use of products with a narrow homolog distribution can be preferred.

Further active products, adjuvants and additives are for example
nonionic polymers such as, for example, vinyl pyrrolidone/vinyl acrylate copolymers, polyvinyl pyrrolidone and vinyl pyrrolidone/vinyl acetate copolymers and polysiloxanes,
cationic polymers such as quaternized cellulose ethers, polysiloxanes with quaternized groups, dimethyidiallylammonium chloride polymers, acrylamide-dimethyldiallylammonium chloride copolymers, dimethylaminoethyl methacrylate-vinyl pyrrolidone copolymers quaternized with diethyl sulfate, vinyl pyrrolidone-imidazolinium methochloride copolymers and quaternized polyvinyl alcohol,
zwitterionic and amphoteric polymers such as, for example, acrylamidopropyltrimethylammonium chloride/acrylate copolymers and octyl acrylamide/methyl methacrylate/tert.-butylaminoethyl methacrylate/2-hydroxypropyl methacrylate copolymers,
anionic polymers such as, for example polyacrylic acid, crosslinked polyacrylic acid, vinyl acetate/crotonic acid copolymers, vinyl pyrrolidone/vinyl acetate copolymers, vinyl acetate/butyl maleate/isobornyl acrylate copolymers, methyl vinyl ether/maleic anhydride copolymers and acrylic acid/ethyl acrylate/N-tert.butylacrylamide terpolymers,
thickeners, such as agar agar, guar gum, alginates, xanthane gum, gum arabic, karaya gum, locust bean flour, linseed gums, dextrans, cellulose derivatives, for example methyl cellulose, hydroxyalkyl cellulose and carboxymethyl cellulose, starch fractions and derivatives, such as amylose, amylopectin and dextrins, clays such as, for example, bentonite or fully synthetic hydrocolloids such as, for example, polyvinyl alcohol,
structurants such as glucose and maleic acid,
hair-conditioning compounds, such as phospholipids, for example soya lecithin, egg lecithin and kephalins, as well as silicone oils,
protein hydrolyzates, particularly hydrolyzates of elastin, collagen, keratin, milk protein, soya protein and wheat protein, their condensation products with fatty acids as well as quaternized protein hydrolyzates,
perfume oils, dimethyl isosorbide and cyclodextrins,
solubilizers such as ethanol, isopropanol, ethylene glycol, propylene glycol, glycerin and diethylene glycol,
anti-dandruff agents, such as piroctone oleamine and zinc omadine,
additional substances to adjust the pH,
active substances such as panthenol, pantothenic acid, allantoin, pyrrolidone carboxylic acids and their salts, plant extracts and vitamins, cholesterol,
light stabilizers
consistency factors, such as sugar esters, polyol esters or polyol alkyl ethers,
fats and waxes, such as spermaceti, beeswax, montan wax and paraffins,
fatty acid alkanolamides,
complexing agents, such as EDTA, NTA and phosphonic acids,
swelling and penetration agents, such as glycerin, propylene glycol monomethyl ether, carbonates, bicarbonates, guanidines, ureas and primary, secondary and tertiary phosphates, imidazoles, tannins, pyrrole,
opacifiers, such as latex,
pearlizers, such as ethylene glycol mono- and distearate,
propellants, such as propane/butane mixtures, N₂O, dimethyl ether, CO₂ and air and
antioxidants.

For the manufacture according to the invention of the suitable dyeing agents, the constituents of the aqueous carrier are added in typical quantities for this application; e.g. emulsifiers are added in concentrations from 0.5 to 30 wt. % and thickeners in concentrations from 0.1 to 25 wt. % of the total dyeing agent.

For the color result it can be advantageous to add ammonium or metal salts to the dyeing agent. Suitable metal salts are e.g. formates, carbonates, halides, sulfates, butyrates, valeriates, capronates, acetates, lactates, glycolates, tartrates, citrates, gluconates, propionates, phosphates and phosphonates of alkali metals, such as potassium, sodium or lithium, earth alkali metals such as magnesium, calcium, strontium or barium, or aluminum, manganese, iron, cobalt, copper or zinc, wherein sodium acetate, lithium bromide, calcium bromide, calcium gluconate, zinc chloride, zinc sulfate, magnesium chloride, magnesium sulfate, ammonium carbonate, -chloride and - acetate are preferred. These salts are preferably comprised in a quantity of 0.03 to 65 mmol, particularly 1 to 40 mmol based on 100 g of the total dyeing agent.

The pH of the ready-to-use dye preparations lies typically between 2 and 12, preferably between 4 and 10.

The CH-acidic compounds according to Formulae C1-C22 and the optionally added color enhancers can be stored either separately or together, either in a liquid to pasty preparation (aqueous or anhydrous) or as dry powder. For separate storage, the components are to be well mixed together immediately prior to their use. For dry storage, normally a defined amount of warm water (30° C. to 80° C.) is added prior to usage and a homogeneous mixture is prepared.

A second subject of the invention is the use of a combination of
(A) at least one oxidizing agent,
(B) at least one CH-acidic compound according to previous Formulae C1-C22,
(C) at least one reactive carbonyl compound
   as a dyeing component in hair dyeing agents.

A third subject of the invention is a process to dye keratin-containing fibers, particularly human hair, wherein a dyeing agent comprising a combination of
(A) at least one oxidizing agent,
(B) at least one CH-acidic compound according to previous Formulae C1-C22,
(C) at least one reactive carbonyl compound
   as well as customary cosmetic ingredients, is applied on keratin-containing fibers for some time, normally about 15-30 minutes, left on the fibers and subsequently rinsed out or washed out with a shampoo.

In doing so, the component B and the component C, particularly their representatives cited above as being preferred and particularly preferred, are applied to the hair as color-providing components either simultaneously or one after the other, i.e., in a multi-step process in which it is unimportant which of the components is applied first. The optionally contained ammonium or metal salts can be added here to the compounds of component B or to the compounds of component C. There can be an interval of up to 30 minutes between the application of the individual components. It is also possible to pretreat the fibers with the salt solution.

In the dyeing process according to the invention, the component A can be applied together with the component B, together with the component C or after the application of components B and C to the hair, so as to form the hair dyeing agent according to the invention. With this aim, the components A and B or A and C can be packaged together in one packaging unit or the component A is stored separately from the components B and C. It is preferred to store the components B and C in different containers or, for separate storage in one box, to use one container having two chambers. However, it is not excluded to store both components B and C together.

During the contact time of the inventive agent on the fibers, it can be advantageous to support the dyeing procedure by providing heat. The heat can be supplied from an external heat source, such as e.g. warm air from a hairdryer or also, particularly with a hair coloration on living test persons, by means of the body temperature of the test persons. The latter option is usually by means of a hood covering over the parts to be dyed.

### EXAMPLES

The following application solutions were prepared:

| Solution 1: | | |
|---|---|---|
| 4-dimethylaminobenzaldehyde | 0.447 | g |
| benzofuran-(2H)-one | 0.402 | g |
| ethanol | 30.000 | g |
| hydrogen peroxide | 1.000 | g |
| ammonia (25% soln.) | to pH 9 | |
| Water | to 100 | |
| Solution 2: | | |
| 4-dimethylaminobenzaldehyde | 0.447 | g |
| benzoylacetonitrile | 0.402 | g |
| ethanol | 30.000 | g |
| hydrogen peroxide | 1.000 | g |
| ammonia (25% soln.) | to pH 9 | |
| water | to 100 | |
| Solution 3: | | |
| 4-dimethylaminobenzaldehyde | 0.447 | g |
| benzofuran-(2H)-one | 0.402 | g |
| ethanol | 30.000 | g |
| hydrogen peroxide | 1.000 | g |
| ammonia (25% soln.) | to pH 9 | |
| water | to 100 | |
| Solution 4: | | |
| 1,3-diiminoisoindoline | 0.435 | g |
| 2-amino-4-imino-2-thiazoline-HCl | 0.454 | g |
| ethanol | 30.000 | g |
| hydrogen peroxide | 1.000 | g |

| | |
|---|---|
| ammonia (25% soln.) | to pH 9 |
| water | to 100 |

A strand of 90% gray, un-pretreated human hair was placed in each freshly prepared dyeing agent solution for 30 minutes at 30° C. The strand was then rinsed with lukewarm water for 30 seconds, dried with warm air (30° C. to 40° C.) and finally combed out.

The resulting color shades and color depths of the color-test examples are presented in the following Table 1.

The color depth was evaluated according to the following scale:

TABLE 1

| Solution | Color Shade | Color Depth |
|---|---|---|
| 1b (inventive) | light brown | (+) |
| 2b (inventive) | bright orange | ++ |
| 3b (inventive) | red brown | ++ |
| 4b (inventive) | Brown orange | +(+) |

− no or a very pale coloration
(+) weak intensity
+ medium intensity
+(+) medium to strong intensity
++ strong intensity
++(+) strong to very strong intensity
+++ very strong intensity

1. An agent for dyeing keratin-containing fibers, comprising:
(A) an oxidizing agent;.
(B) at least one CH— acidic compound selected from compounds according to the following Formulae C1-C22:
compounds with the Formula C1
M¹—CH₂—M² (C1)
in which
M¹ is a group —C(O)M³, —C(O)OM³, —S(O)M³, —SO₂M³, in which M³ stands for a hydrogen atom, an aryl group or a C₁-C₆ alkyl group, or means a group —C(M⁴)═C(C≡N)₂, in which M⁴ stands for a hydrogen atom, a C₁-C₄ alkyl group or an aryl group,
M² has the same meaning as M¹ or is a cyanide group, a substituted or unsubstituted aryl group or aryl-C₁-C₄ alkyl group, a substituted or unsubstituted, saturated or unsaturated heterocycle,
compounds with the Formula C2 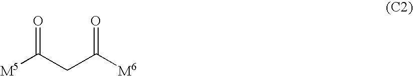
in which
M⁵ is a cyanide group, a substituted or unsubstituted aryl group or an aryl-C₁-C₄ alkyl group, a substituted or unsubstituted, saturated or unsaturated heterocycle or a group —C—O—M⁷ or C(O)OM⁷, in which M⁷ stands for a hydrogen atom or a C₁-C₆ alkyl group,
M⁶ is a substituted or unsubstituted C₁-C₆ alkyl group, an acetyloxy group, a C₃-C₆ cycloalkyl group, a substituted or unsubstituted aryl group or aryl-C₁-C₄ alkyl group, a substituted or unsubstituted aminoaryl group, a substituted or unsubstituted, saturated or unsaturated heterocycle,
compounds with the Formula C3 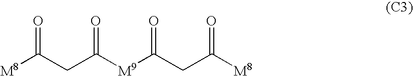
in which
M⁸ is a cyanide group, a substituted or unsubstituted aryl group or aryl-C₁-C₄ alkyl group, a substituted or unsubstituted, saturated or unsaturated heterocycle or a group —COM¹⁰ or COOM¹⁰, in which M¹⁰ stands for a hydrogen atom or a C₁-C₆ alkyl group,
M 9 is a substituted or unsubstituted aryl group or aryl-C₁-C₄ alkyl group, a substituted or unsubstituted aminoaryl group, a substituted or unsubstituted, saturated or unsaturated heterocycle,
pyrazole derivatives (a): selected from the following Formulae C4 and C5 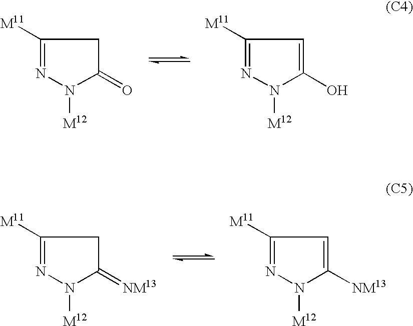
in which
M¹¹ and M¹² independently of one another stand for a substituted or unsubstituted C₁-C₆ alkyl group, a C₃-C₆ cycloalkyl group, a substituted or unsubstituted aryl group or aryl-C₁-C₄ alkyl group, a substituted or unsubstituted aminoaryl group, a substituted or unsubstituted, saturated or unsaturated heterocycle, wherein M¹¹ can additionally mean a C₁-C₆ alkoxy group,
M¹³ is a hydrogen atom or a substituted or unsubstituted C₁-C₆ alkyl group,
(b): two pyrazole rings with the Formulae C4 or C5 linked through M¹¹ or M¹²
barbituric acid derivatives with the Formula C6 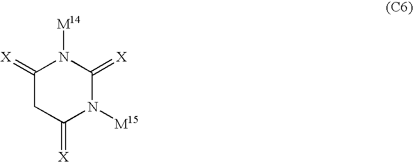
in which
M¹⁴ and M¹⁵ independently of one another stand for a C₁-C₆ alkyl group, a C₂-C₆ alkenyl group, a C₃-C₆ cycloalkyl group, an aryl-C₁-C₄ alkyl group, a substituted or unsubstituted aryl group or a bicyclic group linked through the radicals M¹⁴ or M¹⁵,
X stands for an oxygen or a sulfur atom,
pyridine derivatives with the Formulae C7a and C7b 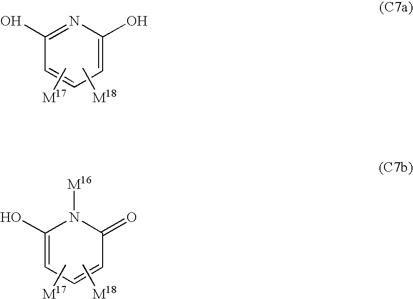
in which
M¹⁶ is a substituted or unsubstituted C₁-C₆ alkyl group or substituted or unsubstituted aryl group,
M¹⁷ is a hydrogen atom, a substituted or unsubstituted C₁-C₆ alkyl group or a substituted or unsubstituted aryl group,
M¹⁸ is a hydrogen atom, a cyanide group, a substituted or unsubstituted C₁-C₆ alkyl group, a group COOM¹⁹, wherein M¹⁹ means a hydrogen atom or a substituted or unsubstituted C₁-C₆ alkyl group,
compounds with the Formula C8 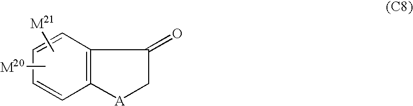
in which
A stands for an oxygen atom, a sulfur atom, a sulfoxyl group, a sulfonyl group or a group NM^{20a}, wherein M^{20a} means a hydrogen atom, a substituted or unsubstituted C₁-C₆ alkyl group,
M²⁰ and M²¹ stand independently from one another for a hydrogen, a fluorine atom, a chlorine atom, a bromine atom, a hydroxyl group, a nitro group, a C₁-C₆ alkyl group, a C₁-C₆ alkoxy, a carboxamide, a sulfonamide, a carboxylic group, a C₁-C₄ acyl, a cyanide group or an amino group —NM²²M²³, in which the M²² and M²³ independently of one another stand for a hydrogen atom or a C₁-C₆ alkyl group,
compounds with the Formulae C9 and C10 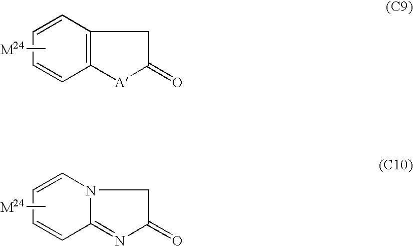
in which
A′ stands for an oxygen atom, a sulfur atom or a group NM²⁵, in which M²⁵ means a hydrogen atom, a substituted or unsubstituted C₁-C₆ alkyl group,
M²⁴ stands for a hydrogen-, fluorine-, chlorine-, a bromine atom, a hydroxyl group, a nitro group, a C₁-C₆ alkyl, a C₁-C₆ alkoxy, a carboxamide, a sulfonamide, a carboxylic group, a C₁-C₄ acyl, a cyanide group or an amino group —NM²⁶M²⁷, in which M²⁶ and M²⁷ independently of one another stand for a hydrogen and a C₁-C₆ alkyl group,
compounds with the Formula C11 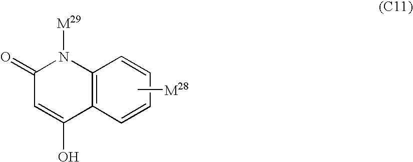
in which
M²⁸ is a hydrogen atom, a halogen atom, a hydroxyl group, a substituted or unsubstituted C₁-C₆ alkyl group, a C₁-C₆ alkoxy group or a nitro group,
M²⁹ stands for a hydrogen atom or a C₁-C₄ alkyl group
indanedione derivatives with the Formula C12 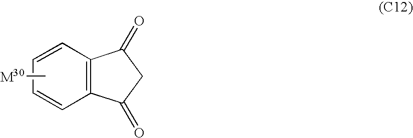
in which
M³⁰ is a hydrogen-, fluorine-, chlorine-, a bromine atom, a nitro group, a C₁-C₆ alkyl, a C₁-C₆ alkoxy, a carboxamide, a sulfonamide or a cyanide group
compounds with the Formula C13 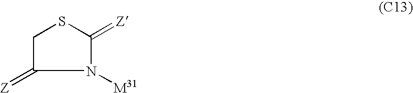
in which
Z stands for an oxygen atom or a group NM³², in which M³² means a hydrogen atom or a C₁-C₆ alkyl group,
Z′ stands for a sulfur atom or a group NM³³, in which M³³ means a hydrogen atom or a C₁-C₆ alkyl group,
M³¹stands for a hydrogen atom, a C₁-C₆ alkyl group or a C₁-C₄ carboxyalkyl group,
dioxopyrazole compounds with the Formula C14 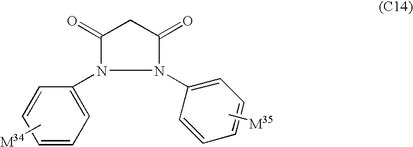
in which
M³⁴ and M³⁵ independently of one another stand for a hydrogen atom, fluorine atom, chlorine atom, a bromine atom, a hydroxyl group, a nitro group, a C₁-C₆ alkyl, a C₁-C₆ alkoxy, a carboxamide, a sulfonamide, a carboxylic group, a C₁-C₄ acyl, a cyanide group or an amino group —NM³⁶M³⁷, in which M³⁶ and M³⁷ independently of one another stand for a hydrogen and a C₁-C₆ alkyl group,
5-oxoimidazole derivatives with the Formula C15 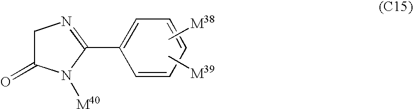
in which
M³⁸ and M³⁹ independently of one another stand for a hydrogen atom, a fluorine atom, a chlorine atom, a bromine atom, a hydroxyl group, a nitro group, a C₁-C₆ alkyl, a C₁-C₆ alkoxy, a carboxamide, a sulfonamide, a carboxylic group, a C₁-C₄ acyl, a cyanide group or an amino group —NM⁴¹M⁴², in which M⁴¹ and M⁴² independently of one another stand for a hydrogen atom or a C₁-C₆ alkyl group,
M⁴⁰ stands for a hydrogen atom or a C₁-C₆ alkyl group,
derivatives of dehydrobutyrolactone with the Formula C16 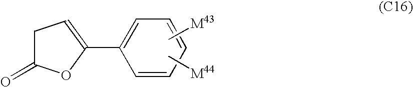
in which
M⁴³ and M⁴⁴ independently of one another stand for a hydrogen atom, a fluorine atom, a chlorine atom, a bromine atom, a hydroxyl group, a nitro group, a C₁-C₆ alkyl, a C₁-C₆ alkoxy, a carboxamide, a sulfonamide, a carboxylic group, a C₁-C₄ acyl, a cyanide group or an amino group —NM⁴⁵M⁴⁶, in which M⁴⁵ and M⁴⁶ independently of one another stand for a hydrogen atom or a C₁-C₆ alkyl group,
compounds with the Formula C17 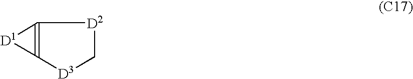
in which
D¹ is a condensed aromatic or heteroatomic ring,
D² is a carbonyl group, a group C═CD^{I}D^{II} or a group CD^{I}D^{II}, in which D^{I} and D^{II} each have a substituent with a Hammett Constant between 0.4 and 2.0, or both substituents add up to a Hammett Constant between 0.4 and 2.0;
D³ stands for a carbonyl group, an oxygen atom, a sulfur atom, a group NM⁴⁷, when D² is not oxygen, or a group C═S, a group C═NR⁴⁸, a sulfinyl group, a sulfonyl group, wherein R⁴⁷ and R⁴⁸ independently of one another mean a hydrogen atom or a C₁-C₄ alkyl radical,
pyrimidine derivatives with the Formula C18 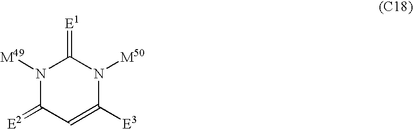
in which
M⁴⁹ and M⁵⁰ independently of one another are a hydrogen atom or a substituted or unsubstituted C₁-C₆ alkyl group,
E¹ stands for an oxygen, a sulfur atom or a NH group,
E² stands for a NH group or an oxygen atom,
E³ stands for an amino group or a hydroxyl group,
with the proviso, that
c) when E¹ and E² stand for an oxygen atom, E³ is not a hydroxyl group, and
d) when E¹ is a sulfur atom and E² is an oxygen atom, E³ is not a hydroxyl group,
quaternized nitrogen compounds of Formula C19 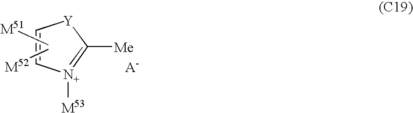
in which,
M⁵¹ and M⁵² independently of one another stand for a hydrogen atom, a halogen atom, a hydroxyl group, a C₁-C₄ hydroxyalkyl group, a C₁-C₆ aminoalkyl group, a C₁-C₄ dialkylamino-C₁-C₄ alkyl group, a linear or branched C₁-C₆ alkyl group, a C₂-C₆ alkenyl group, an optionally substituted aryl group, a sulfonic acid group, a carboxylic group, a formyl group, a nitro group, a cyanide group or a group —NM⁵⁴M⁵⁵, wherein M⁵⁴ and M⁵⁵ independently of one another stand for a hydrogen atom, a C₁-C₆ alkyl group, a C₂-C₆ alkenyl group, an aryl C₁-C₄ alkyl group or a C₁-C₄ hydroxyalkyl group, wherein M⁵¹ and M⁵² can together form a condensed 5 or 6 membered aliphatic or aromatic or heteroaromatic ring, which is again substituted with the radicals M⁵⁶ and M⁵⁷, wherein M⁵⁶ and M⁵⁷ independently of one another stand for the radicals defined for M⁵¹,
M⁵³ stands for a hydrogen atom, a C₁-C₄ hydroxyalkyl group, a C₁-C₆ aminoalkyl group, a C₁-C₄ dialkylamino-C₁-C₄ alkyl group, a linear or branched C₁-C₆ alkyl group, a C₂-C₆ alkenyl group, an optionally substituted aryl group, a C₁-C₄ sulfoalkyl group, a C₁-C₄ carboxyalkyl group or a C₂-C₆ polyhydroxyalkyl group,
Y stands for an oxygen atom, a sulfur atom, an optionally substituted methylene group or a group NM⁶⁰, wherein M⁶⁰ can stand for the same groups that are defined for M⁵⁵,
A⁻ stands for a chloride, bromide, iodide, hexafluorophosphate, tetrachlorozincate, tetrafluoroborate, trifluoromethyl sulfonate, methyl sulfonate or p-toluene sulfonate,
onium compounds with the Formulae C20 and C21 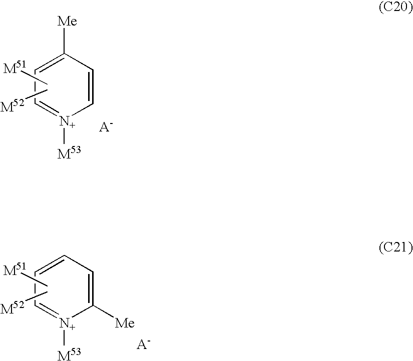
wherein M⁵¹, M⁵², M⁵³ and A⁻ are selected from the groups that are defined for compound C19.
compounds with the general Formula C22 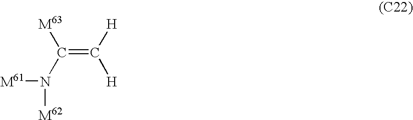
in which
M⁶¹ stands for an aromatic radical, particularly for a 5- or 6-membered aryl radical optionally substituted with a C₁-C₄ alkyl, C₁-C₄ hydroxyalkyl, hydroxy, methoxy or halide group, preferably a phenyl radical, or a 5- or 6-membered, condensed, aliphatic or aromatic, carbocyclic or heterocyclic ring preferably a phenyl radical, a quinoline radical or a pyridyl radical.
M⁶² stands for a hydrogen atom, a linear or branched C₁-C₈ alkyl group, a linear or branched C₁-C₈ hydroxyalkyl group or a C₁-C₈ alkoxyalkyl group, wherein an oxygen atom can be between the C atoms of the alkyl chain, and
M⁶³ stands for a linear or branched C₁-C₈ alkyl group, a C₁-C₆ alkoxyalkyl C₁-C₆ alkyl group, a C₁-C₆ alkylamino C₁-C₆ alkyl group, a C₁-C₆ alkylmercapto C₁-C₆ alkyl group, a C₁-C₆ alkoxy C₁-C₆ alkylene group, a C₁-C₆ alkylamino C₁-C₆ alkylene group, a C₁-C₆ alkylmercapto C₁-C₆ alkylene group, a straight chain or branched C₁-C₈ alkylene group or an oxygen atom, a nitrogen atom or a sulfur atom, with the proviso that the radicals M⁶¹ and M⁶³, together with the nitrogen atom and the carbon atom of the basic enamine structure, form a cyclic compound, when M⁶³ equals a linear or branched C₁-C₈ alkylene group, a C₁-C₈ alkoxyalkylene group, a C₁-C₆ alkylamino C₁-C₆ alkylene group, a C₁-C₆ alkylmercapto C₁-C₆ alkylene group, an oxygen atom, a nitrogen atom or a sulfur atom;
(C) at least one reactive carbonyl compound.

2. The agent according to claim 1, wherein component A is selected from the group consisting of H₂O₂ and H₂O₂ generating compounds.

3. The agent according to claim 1, wherein component A is a H₂O₂ generating compound selected from the group consisting of sodium carbonate peroxy hydrate, urea peroxide and melamine perhydrate.

4. The agent according to claim 1, wherein the oxidizing agent is added in a quantity of 0.01 to 6 wt. %, based on the applied solution.

5. The agent according to claim 1, characterized in that component A is H₂O₂ used in combination with a compound selected from the group consisting of magnesium peroxide, sodium peroxydisulfate, potassium peroxydisulfate, ammonium peroxydisulfate, sodium peroxydiphosphonate, potassium peroxydiphosphonate, and ammonium peroxydiphosphonate.

6. The agent according to claim 1, wherein component B comprises at least one compound selected from the group consisting of benzofuran-(2H)-one, benzoylacetonitrile and 2-amino-4-imino-2-thiazoline and their physiologically acceptable salts.

7. The agent according to claim 1, wherein the reactive carbonyl compounds of component C are selected from the group which is formed from the compound according to Formulae I, IV, V, Vla or Vlb, 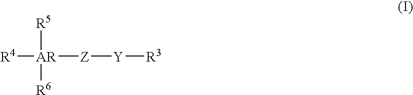
wherein
AR stands for benzene, naphthalene, pyridine, pyrimidine, pyrazine, pyridazine, carbazole, pyrrole, pyrazole, furan, thiophene, 1,2,3-triazine, 1,3,5-triazine, quinoline, isoquinoline, indole, indoline, indolizine, indane, imidazole, 1,2,4-triazole, 1,2,3-triazole, tetrazole, benzimidazole, 1,3-thiazole, benzothiazole, indazole, benzoxazole, quinoxaline, quinazoline, quinolizine, cinnoline, acridine, julolidine, acenaphthene, fluorene, biphenyl, diphenylmethane, benzophenone, diphenyl ether, azobenzene, chromone, coumarine, diphenylamine, stilbene, wherein the N-heteroaromatics can also be quaternized,
R³ stands for a hydrogen atom, a C₁-C₆ alkyl, C₂-C₆ acyl, C₂-C₄ alkenyl, C₁-C₄ perfluoroalkyl, an optionally substituted aryl or heteroaryl group,
R⁴, R⁵ and R⁶ independently from one another stand for a hydrogen atom, a halogen atom, a C₁-C₆ alkyl, C₁-C₆ alkoxy, C₁-C₆ aminoalkyl, C₁-C₆ hydroxyalkyl group, a C₁-C₆ alkoxy-C₁-C₆ alkyloxy group, a C₂-C₆ acyl group, an acetyl, a carboxyl, carboxylato, carbamoyl, sulfo, sulfato, sulfonamide, sulfonamido, C₂-C₆ alkenyl, an aryl, an aryl C₁-C₆ alkyl group, a hydroxyl, a nitro, a pyrrolidino, a morpholino, a piperidino, an amino or ammonio or a 1-imidazol(in)io group, wherein the last three groups can be substituted with one or more C₁-C₆ alkyl-, C₁-C₆ carboxyalkyl-, C₁-C₆ hydroxyalkyl, C₂-C₆ alkenyl, C₁-C₆ alkoxy-C₁-C₆ alkyl, with optionally substituted benzyl groups, with sulfo-(C₁-C₄)-alkyl or heterocycle-(C₁-C₄) alkyl groups,
wherein also two of the radicals R⁴, R⁵, R⁶ and -Z—Y—R³, together with the remainder of the molecule, can form a condensed, optionally substituted 5-, 6- or 7-membered ring that can equally have a condensed aromatic ring on it, wherein the system AR, depending on the ring size, can have further substituents that independently of one another can stand for the same groups as R⁴, R⁵ and R⁶,
Z stands for a direct bond, a carbonyl, a carboxy-(C₁-C₄) alkylene, an optionally substituted C₂-C₆ alkenylene, C₄-C₆ alkadienylene, furylene, thienylene, arylene, vinylenearylene, vinylenefurylene, vinylenethienylene group, wherein Z, together with the —Y—R³ group can also form an optionally substituted 5-, 6- or 7-membered ring,
Y stands for a group selected from carbonyl, a group according to Formula II and a group according to Formula III, 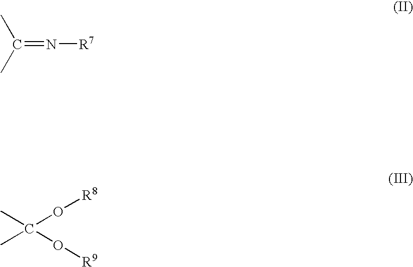
wherein
R⁷ stands for a hydrogen atom, a hydroxyl group, a C₁-C₄ alkoxy group, a C₁-C₆ alkyl group, a C₁-C₆ hydroxyalkyl group, a C₂-C₆ polyhydroxyalkyl group, a C₁-C₆ alkoxy-C₁-C₆ alkyl group,
R⁸ and R⁹ independently of one another stand for a C₁-C₆ alkyl group, an aryl group or form, together with the structural element O—C—O of Formula III, a 5- or 6-membered ring.
aldehydes with the Formula (IV) 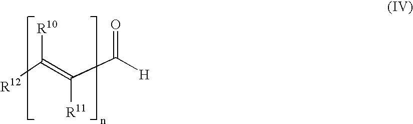
in which
R¹⁰ and R¹¹ independently of one another stand for a hydrogen atom, a C₁-C₆ alkyl group, a C₁-C₄ hydroxyalkyl group, a C₂-C₈ polyhydroxyalkyl group, a C₁-C₄ alkoxy C₁-C₄ alkyl group, a C₁-C₄ alkoxy group, a group —OCF₃ or a group —CF₃, wherein the radicals R¹⁰ and R¹¹, together with the vinylene moiety in the brackets can form a 5- or 6-membered, aromatic or aliphatic, carbocyclic or heterocyclic ring,
n equals 0, 1, 2 or 3
R¹² stands for a hydrogen atom, a C₁-C₆ alkyl group, a C₁-C₄ hydroxyalkyl group, a C₂-C₈ polyhydroxyalkyl group, a C₁-C₄ alkoxy C₁-C₄ alkyl group, a C₁-C₄ alkoxy group, a group —OCF₃ or a group —CF₃,
ketones with the Formula (V) 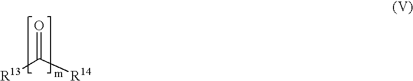
in which
R¹³ stands for a hydrogen atom, a C₁-C₆ alkyl group, a C₁-C₄ hydroxyalkyl group, a C₂-C₈ polyhydroxyalkyl group, a C₁-C₄ alkoxy C₁-C₄ alkyl group, a C₁-C₄ alkoxy group, a group —OCF₃ or a group —CF₃,
R¹⁴ stands for a C₁-C₆ alkyl group, a C₁-C₄ monohydroxyalkyl group, a C₂-C₈ polyhydroxyalkyl group,
m equals 1 or 2
wherein R¹³ and R¹⁴, together with the carbonyl moiety can form a 5- or 6-membered, aliphatic or aromatic, carbocyclic or heterocyclic ring that optionally again carries a condensed 5- or 6-membered, aliphatic or aromatic, carbocyclic or heterocyclic ring, with the proviso that each heterocyclic ring comprises nitrogen or sulfur atoms as heteroatoms,
diimino-isoindoline- or 3-amino-isoindoline derivatives according to Formulae Vla and Vlb, 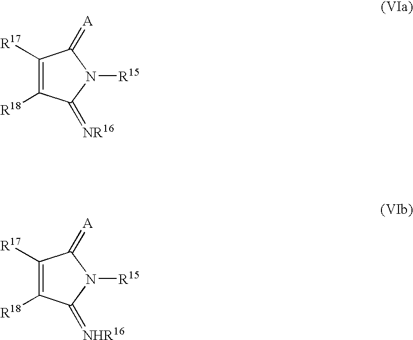
in which
A stands for an oxygen atom or a NH group,
R¹⁵ and R¹⁶ independently of one another stand for a hydrogen atom, a C₁-C₄ alkyl group, a C₁-C₄ hydroxyalkyl group, a C₂-C₆ polyhydroxyalkyl group, a group R^{I} R^{II}N—(CH₂)_{q}—, wherein R^{I} and R^{II} independently of one another stand for a C₁-C₆ alkyl group or an aryl C₁-C₆ alkyl group and q stands for a number 1, 2, 3, 4, 5 or 6, wherein R^{I} and R^{II}, together with the nitrogen atom, can form an aromatic or aliphatic 5-, 6- or 7-membered heterocycle,
R¹⁷ and R¹⁸, together with the remainder of the molecule, can form a 5- or 6-membered, aromatic or heteroaromatic ring that can optionally be substituted.

8. The agent according to claim 1, characterized in that component C comprises at least one compound selected from the group consisting of
5-(4-dimethylaminophenyl)penta-2,4-dienal, 5-(4-diethylaminophenyl)penta-2,4-dienal, 5-(4-methoxyphenyl)penta-2,4-dienal, 5-(3,4-dimethoxyphenyl)penta-2,4-dienal, 5-(2,4-dimethoxyphenyl)penta-2,4-dienal, 5-(4-piperidinophenyl)penta-2,4-dienal, 5-(4-morpholinophenyl)penta-2,4-dienal, 5-(4-pyrrolidinophenyl)penta-2,4-dienal, 6-(4-dimethylaminophenyl)hexa-3,5-dien-2-one, 6-(4-diethylaminophenyl)hexa-3,5-dien-2-one, 6-(4-methoxyphenyl)hexa-3,5-dien-2-one, 6-(3,4-dimethoxyphenyl)hexa-3,5-dien-2-one, 6-(2,4-dimethoxyphenyl)hexa-3,5-dien-2-one, 6-(4-piperidinophenyl)hexa-3,5-dien-2-one, 6-(4-morpholinophenyl)hexa-3,5-dien-2-one, 6-(4-pyrrolidinophenyl)hexa-3,5-dien-2-one, 5-(4-dimethylaminonaphth-1-yl)penta-2,4-dienal,
2-nitropiperonal, 5-nitropiperonal, 6-nitropiperonal, 5-hydroxy-2-nitropiperonal, 2-hydroxy-5-nitropiperonal, 2-chloro-6-nitropiperonal, 5-chloro-2-nitropiperonal, 2,6-dinitropiperonal,
2-nitrobenzaldehyde, 3-nitrobenzaldehyde, 4-nitrobenzaldehyde, 4-methyl-3-nitrobenzaldehyde, 3-hydroxy-4-nitrobenzaldehyde, 4-hydroxy-3-nitrobenzaldehyde, 5-hydroxy-2-nitrobenzaldehyde, 2-hydroxy-5-nitrobenzaldehyde, 2-hydroxy-3-nitrobenzaldehyde, 2-fluoro-3-nitrobenzaldehyde, 3-methoxy-2-nitrobenzaldehyde, 4-chloro-3-nitrobenzaldehyde, 2-chloro-6-nitrobenzaldehyde, 5-chloro-2-nitrobenzaldehyde, 4-chloro-2-nitrobenzaldehyde, 2,4-dinitrobenzaldehyde, 2,6-dinitrobenzaldehyde, 2-hydroxy-3-methoxy-5-nitrobenzaldehyde, 4,5-dimethoxy-2-nitrobenzaldehyde, 5-nitrovanillin, 3,5-dinitrosalicylaldehyde, 5-bromo-3-nitrosalicylaldehyde, 3-nitro-4-formylbenzenesulfonic acid, 4-nitro-1-naphthaldehyde, 2-nitrocinnamaldehyde, 3-nitrocinnamaldehyde, 4-nitrocinnamaldehyde, carbazole aldehydes or carbazole ketones, particularly 9-methyl-3—
carbazolealdehyde, 9-ethyl-3-carbazolealdehyde, 3-acetylcarbazole, 3,6-diacetyl-9-ethylcarbazole, 3-acetyl-9-methylcarbazole, 1,4-dimethyl-3-carbazole aldehyde, 1,4,9-trimethyl-3-carbazolealdehyde,
4-trimethylammoniobenzaldehyde-, 4-benzyldimethylammoniobenzaldehyde-, 4-trimethylammoniocinnamaldehyde-, 4-trimethylammonionaphthaldehyde-, 2-methoxy-4-trimethylammoniobenzaldehyde-, N-(4-acetylphenyl)-trimethylammonium-, 4-(N,N-Diethyl)-N-methylammonio)-benzaldehyde-, N-(4-benzoylphenyl)-trimethylammonium-, N-(4-benzoylphenyl)-N,N-diethylmethylammonium-, N-(4-formylphenyl)-N-methylpyrrolidinium-, N-(4-formylphenyl)-N-methylpiperidinium-, N-(4-formylphenyl)-N-methylmorpholinium-, N-(4-acetylphenyl)-N-methylmorpholinium-, N(4-benzoylphenyl)-N-methylmorpholinium-, 3-formyl-N-ethyl-N-methylcarbazolium-, 3-formyl-9,9-dimethylcarbazolium-, 1-(4-acetylphenyl)-3-methylimidazolium-, 1-(4-acetylphenyl)-3-methyl-2-imidazolinium-, 1-(4-benzoylphenyl)-3-methylimidazolium-, 5-acetyl-1,3-diethyl-2-methylbenzimidazolium-, 5-trimethylammonio-1-indanone-salts, particularly the benzenesulfonates, p-toluenesulfonates, methanesulfonates, ethanesulfonates, propanesulfonates, perchlorates, sulfates, chlorides, bromides, iodides, tetrachlorozincates, methylsulfates, trifluoromethanesulfonates, hexafluorophosphates, tetrafluoroborates,
4-formyl-1-methylpyridinium-, 2-formyl-1-methylpyridinium-, 4-formyl-1-ethylpyridinium-, 2-formyl-1-ethylpyridinium-, 4-formyl-1-benzylpyridinium-, 2-formyl-1-benzylpyridinium-, 4-formyl-1,2-dimethylpyridinium-, 4-formyl-1,3-dimethylpyridinium-, 4-formyl-1-methylquinolinium-, 2-formyl-1-methylquinolinium-, 4-acetyl-1-methylpyridinium-, 2-acetyl-1-methylpyridinium-, 4-acetyl-1-methylquinolinium-, 5-formyl-1-methylquinolinium-, 6-formyl-1-methylquinolinium-, 7-formyl-1-methylquinolinium-, 8-formyl-1-methylquinolinium, 5-formyl-1-ethylquinolinium-, 6-formyl-1-ethylquinolinium-, 7-formyl-1-ethylquinolinium-, 8-formyl-1-ethylquinolinium, 5-formyl-1-benzylquinolinium-, 6-formyl-1-benzylquinolinium-, 7-formyl-1-benzylquinolinium-, 8-formyl-1-benzylquinolinium, 5-formyl-1-allylquinolinium-, 6-formyl-1-allylquinolinium-, 7-formyl-1-allylquinolinium- and 8-formyl-1-allylquinolinium-, 5-acetyl-1-methylquinolinium-, 6-acetyl-1-methylquinolinium-, 7-acetyl-1-methylquinolinium-, 8-acetyl-1-methylquinolinium, 5-acetyl-1-ethylquinolinium-, 6-acetyl-1-ethylquinolinium-, 7-acetyl-1-ethylquinolinium-, 8-acetyl-1-ethylquinolinium, 5-acetyl-1-benzylquinolinium-, 6-acetyl-1-benzylquinolinium-, 7-acetyl-1-benzylquinolinium-, 8-acetyl-1-benzylquinolinium, 5-acetyl-1-allylquinolinium-, 6-acetyl-1-allylquinolinium-, 7-acetyl-1-allylquinolinium- and 8-acetyl-1-allylquinolinium, 9-formyl-10-methylacridinium-, 4-(2-formylvinyl)-1-methylpyridinium-, 1,3-dimethyl-2-(4-formylphenyl)-benzimidazolinium-, 1,3-dimethyl-2-(4-formylphenyl)-imidazolinium-, 2-(4-formylphenyl)-3-methylbenzothiazolium-, 2-(4-acetylphenyl)-3-methylbenzothiazolium-, 2-(4-formylphenyl)-3-methylbenzoxazolium-, 2-(5-formyl-2-furyl)-3-methylbenzothiazolium-, 2-(5-formyl-2-thienyl)-3-methylbenzothiazolium-, 2-(3-formylphenyl)-3-methylbenzothiazolium-, 2-(4-formylnaphth-1-yl)-3-methylbenzothiazolium-, 5-chloro-2-(4-formylphenyl)-3-methylbenzothiazolium-, 2-(4-formylphenyl)-3,5-dimethylbenzothiazolium-, 1-methyl-2-[2-(4-formylphenyl)-ethenyl]pyridinium-, 1-methyl-4-[2-(4-acetylphenyl)-ethenyl]-pyridinium-, 1-benzyl-4-[2-(4-formylphenyl)-ethenyl]-pyridinium-, 1-methyl-4-[2-(4-formylphenyl)-ethenyl)pyridinium-, 1-methyl-2-[2-(4-formylphenyl)-ethenyl]pyridinium-, 1-methyl-4-[2-(4-formylphenyl)-ethenyl] quinolinium-, 1-methyl-2-[2-(4-formylphenyl)-ethenyl]-quinolinium-, 1-methyl-2-[2-(5-formyl-2-furyl)-ethenyl]-quinolinium-, 1-methyl-2-[2-(5-formyl-2-thienyl)-ethenyl]-quinolinium-, 1-methyl-2-[2-(4-formylphenyl)-ethenyl]benzothiazolinium-, 1,3-dimethyl-2-[2-(4-formylphenyl)-ethenyl]-benzimidazolinium-, 1,3-dimethyl-2-[2-(4-formylphenyl)-ethenyl]-imidazolinium-, 1-methyl-5-oxo-indeno[1,2-b]pyridinium(4-methyl-4-azonio-9-fluorenon-), 1-ethyl-5-oxo-indeno[1,2-b]pyridinium(4-ethyl-4-azonio-9-fluorenon-), 1-benzyl-5-oxoindeno[1,2-b]pyridinium(4-benzyl-4-azonio-9-fluorenon-), 2-methyl-5-oxoindeno[1,2-c]pyridinium-, 2-methyl-9-oxo-indeno[2, 1-c]pyridinium-, 1-methyl-9-oxoindeno[2, 1-b]pyridinium-salts, particularly benzenesulfonate, p-toluenesulfonate, methanesulfonate, perchlorate, sulfate, chloride, bromide, iodide, tetrachlorozincate, methylsulfate, trifluoromethanesulfonate, tetrafluoroborate,
salicylaldehyde, vanillin, 4-hydroxy-3-methoxycinnamaldehyde (coniferylaldehyde), 2,4-dihydroxybenzaldehyde, 4-dimethylaminobenzaldehyde, 4-diethylaminobenzaldehyde, 4-dimethylamino-2-hydroxybenzaldehyde, 4-pyrrolidinobenzaldehyde, 4-morpholinobenzaldehyde, 4-piperidinobenzaldehyde, 4-dimethylaminoacetophenone, 4-hydroxynaphthaldehyde, 4-dimethylaminonaphthaldehyde, 4-dimethylaminobenzylidenacetone, 4-dimethylaminozimtaldehyde, 2-dimethylaminobenzaldehyde, 2-chloro-4-dimethylaminobenzaldehyde, 4-dimethylamino-2-methylbenzaldehyde, trans-4-diethylamino-cinnamaldehyde, 4-(dibutylamino)-benzaldehyde, 4-diphenylaminobenzaldehyde, 2,3,6,7-tetrahydro-1H,5H-benzo[ij]quinolizine-9-carboxaldehyde, 4-dimethylamino-2-methoxybenzaldehyde, 2,3,6,7-tetrahydro-8-hydroxy-1H,5H-benzo[ij]quinolizine-9-carboxaldehyde, 4-(1-imidazolyl)-benzaldehyde, 2-morpholinobenzaldehyde, indole-3-carboxaldehyde, 1-methylindole-3-carboxaldehyde, N-ethylcarbazole-3-carboxaldehyde, 2-formylmethylene-1,3,3-trimethylindoline (tribasic aldehyde)
1,3-diacetylbenzene, 1,4-diacetylbenzene, 1,3,5-triacetylbenzene, benzoyl-acetophenone, 2-(4′-methoxybenzoyl)acetophenone, 2-(2′-furoyl)acetophenone, 2-(2′-pyridoyl)acetophenone, 2-(3′-pyridoyl)acetophenone,
1-phenyl-1,2-propanedione, 1-phenyl-1,2-butanedione, 1-phenyl-3,3-dimethyl-1,2-butanedione, benzil, anisil, salicil, 5,5′-dibromosalicil, 2,2′-furil, 2,2′-thienil, 2,2′-, 4,4′-pyridil, 6,6′-dimethyl-4,4′-pyridil, 4-hydroxy-, 4-methoxy-, 4-chloro-, 4-methyl-, 4-dimethylamino-, 4,4′-dihydroxy-, -dimethyl-, -dibromo-, -dichloro-, -bisdimethylamino-, 2,4-dihydroxy-, 3,3′-dimethoxy, 2′-chloro-3,4-dimethoxy-, 3,4,5,3′,4′,5′-hexamethoxybenzil, isatin derivatives, such as 5-chloroisatin, 5-methoxyisatin, 5-nitroisatin,
6-nitroisatin, 5-sulfoisatin, isatin-5-sulfonic acid, isatin-4-carboxylic acid and isatin-5-carboxylic acid,
N-substituted isatin derivatives, such as N-methylisatin, N-(2-hydroxyalkyl)-isatin, N-(2-hydroxypropyl)-isatin, N-(3-hydroxypropyl)-isatin, N-(2,3-dihydroxypropyl)-isatin, N-(2-sulfoethyl)-isatin, (3-sulfopropyl)-isatin, N-allylisatin, N-vinylisatin, N-benzylisatin, N-(4-methoxybenzyl)-isatin, N-(4-carboxybenzyl)-isatin, N-(4-sulfobenzyl)-isatin, N-(2-dimethylaminoethyl)-isatin, N-(2-pyrrolidinoethyl)-isatin, N-(2-piperidinoethyl)-isatin, (2-morpholinoethyl)-isatin, N-(2-furylmethyl)-isatin, n(thien-2-ylmethyl)-isatin, N-(pyrid-2-ylmethyl)-isatin, N-(pyrid-3-ylmethyl)-isatin, N-(pyrid-4-ylmethyl)-isatin, N-allylisatin-5-sulfonic acid 5-chloro-N-(2-hydroxyethyl)isatin, 5-methyl-N-(2-hydroxyethyl)-isatin, 5,7-dichloro-N-allylisatin, 5-nitro-N-allylisatin, N-hydroxymethylisatin, N-hydroxymethyl-5-methylisatin, N-hydroxymethyl-5-chloroisatin, N-hydroxymethyl-5-sulfoisatin, N-hydroxymethyl-5-carboxyisatin, N-hydroxymethyl-5-nitroisatin, N-hydroxymethyl-5-bromoisatin, N-hydroxymethyl-5-methoxyisatin, N-hydroxymethyl-5,7-dichloroisatin, N-dimethylaminomethylisatin, N-diethylaminomethylisatin, N-(bis(2-hydroxyethyl)-aminomethyl)-isatin, N-(2-hydroxyethylaminomethyl)-isatin, N-(bis-(2-hydroxypropyl)aminomethyl)-isatin, N-pyrrolidinomethylisatin, N-piperidinomethylisatin, N-morpholinomethylisatin, N-(1,2,4-triazol-1-ylmethyl)isatin, N-(imidazol-1-ylmethyl)isatin, N-carboxymethylaminomethylisatin, N-(2-carboxyethylaminomethyl)-isatin, N-(3-carboxypropylaminomethyl)-isatin, N-(bis(2-hydroxyethyl)-aminomethyl)-5-methylisatin, N-piperidinomethyl-5-chloroisatin, N-(2-sulfoethylamino)-isatin, as well as the alkali- and optional ammonium salts of the acidic compounds, quinisatin and their derivatives, such as N-methylquinisatin,
acetophenone, propiophenone, 2-hydroxyacetophenone, 3-hydroxyacetophenone, 4-hydroxyacetophenone, 2-hydroxypropiophenone, 3-hydroxypropiophenone, 4-hydroxypropiophenone, 2-hydroxybutyrophenone, 3-hydroxybutyrophenone, 4-hydroxybutyrophenone, 2,4-dihydroxyacetophenone, 2,5-dihydroxyacetophenone, 2,6-dihydroxyacetophenone, 2,3,4-trihydroxyacetophenone, 3,4,5-trihydroxyacetophenone, 2,4,6-trihydroxyacetophenone, 2,4,6-trimethoxyacetophenone, 3,4,5-trimethoxyacetophenone, 3,4,5-trimethoxy-acetophenone-diethylketal, 4-hydroxy-3-methoxy-acetophenone, 3,5-dimethoxy-4-hydroxy-acetophenone, 4-amino-acetophenone, 4-dimethylamino-acetophenone, 4-morpholino-acetophenone, 4-piperidinoacetophenone, 4-imidazolino-acetophenone, 2-hydroxy-5-bromo-acetophenone, 4-hydroxy-3-nitroacetophenone, acetophenone-2-carboxylic acid, acetophenone-4-carboxylic acid, benzophenone, 4-hydroxybenzophenone, 2-amino-benzophenone, 4,4′-dihydroxybenzophenone, 2,4-dihydroxybenzophenone, 2,4,4′-trihydroxybenzophenone, 2,3,4-trihydroxybenzophenone, 2-hydroxy-1-acetonaphthone, 1-hydroxy-2-acetonaphthone, chromone, chromone-2-carboxylic acid, flavone, 3-hydroxyflavone, 3,5,7-trihydroxyflavone, 4′,5,7-trihydroxyflavone, 5,6,7-trihydroxyflavone, quercetin, indanone, 9-fluorenone, 3-hydroxyfluorenone, anthrone, 1,8-dihydroxyanthrone,
heterocyclic carbonyl compounds, such as 2-indolaldehyde, 3-indolaldehyde, 1-methylindol-3-aldehyde, 2-methylindol-3-aldehyde, 1-acetylindol-3-aldehyde, 3-acetylindole, 1-methyl-3-acetylindole, 2-(1,3,3-trimethyl-2-indolinylidene)-acetaldehyde, 1-methylpyrrol-2-aldehyde, 1-methyl-2-acetylpyrrole, 1-pyridinealdehyde, 2-pyridinealdehyde, 3-pyridinealdehyde, 4-acetylpyridine, 2-acetylpyridine, 3-acetylpyridine, pyridoxal, quinoline-3-aldehyde, quinoline-4-aldehyde, antipyrine-4-aldehyde, furfural, 5-nitrofurfural, 2-thenoyl-trifluoroacetone, chromone-3-aldehyde, 3-(5-nitro-2-furyl)acrolein, 3-(2-furyl)acrolein, imidazole-2-aldehyde, 1,3-diimino-isoindoline,
indanone derivatives, such as e.g. 1,2-indandione, 2-oximo-1-indanone, indan-1,2,3-trione-2-oxime, 5-methoxy-indan-1,2,3-trione-2-oxime, 2-nitro-1,3-indandione
as well as physiologically acceptable salts of the above-mentioned compounds.

9. The agent according to claim 1, additionally comprising component D selected from the group consisting of (a) compounds with primary or secondary amino groups or hydroxyl groups selected from primary and secondary aromatic amines, nitrogen-containing heterocyclic compounds and aromatic hydroxy compounds, (b) amino acids, (c) oligopeptides constructed from 2 to 9 amino acids and the physiologically acceptable salts of (a)-(c).

10. The agent according to claim 9, characterized in that the primary and secondary aromatic amines of component D are selected from the group consisting of N,N-dimethyl-p-phenylenediamine, N,N-diethyl-p-phenylenediamine, N-(2-hydroxyethyl)-N-ethyl-p-phenylenediamine, N ,N-bis-(2-hydroxyethyl)-p-phenylenediamine, N-(2-methoxyethyl)-p-phenylenediamine, 2,3-dichloro-p-phenylenediamine, 2,4-dichloro-p-phenylenediamine, 2,5-dichloro-p-phenylenediamine, 2-chloro-p-phenylenediamine, 2,5-dihydroxy-4-morpholinoaniline, 2-aminophenol, 3-aminophenol, 4-aminophenol, 2-aminomethyl-4-aminophenol, 2-hydroxymethyl-4-aminophenol, o-phenylenediamine, m-phenylenediamine, p-phenylenediamine, 2,5-diaminotoluene, 2,5-diaminophenol, 2,5-diaminoanisole, 2,5-diaminophenethol, 4-amino-3-methylphenol, 2-(2,5-diaminophenyl)ethanol, 2,4-diaminophenoxyethanol, 2-(2,5-diaminophenoxy)ethanol, 3-amino-4-(2-hydroxyethyloxy)phenol, 3,4-methylenedioxyphenol, 3,4-methylenedioxyaniline, 3-amino-2,4-dichlorophenol, 4-methylaminophenol, 2-methyl-5-aminophenol, 3-methyl-4-aminophenol, 2-methyl-5-(2-hydroxyethylamino)phenol, 3-amino-2-chloro-6-methylphenol, 2-methyl-5-amino-4-chlorophenol, 5-(2-hydroxyethylamino)-4-methoxy-2-methylphenol, 4-amino-2-hydroxymethylphenol, 2-(diethylaminomethyl)-4-aminophenol, 4-amino-1-hydroxy-2-(2-hydroxyethylaminomethyl)benzene, 1-hydroxy-2-amino-5-methyl-benzene, 1-hydroxy-2-amino-6-methylbenzene, 2-amino-5-acetamidophenol, 1,3-dimethyl-2,5-diaminobenzene, 5-(3-hydroxypropylamino)-2-methylphenol, 5-amino-4-methoxy-2-methylphenol, N,N-dimethyl-3-aminophenol, N-cyclopentyl-3-aminophenol, 5-amino-4-fluoro-2-methylphenol, 2,4-diamino-5-fluorotoluene, 2,4-diamino-5-(2-hydroxyethoxy)-toluene, 2,4-diamino-5-methylphenetol, 3,5-diamino-2-methoxy-1-methylbenzene, 2-amino-4-(2-hydroxyethylamino)anisole, 2,6-bis-(2-hydroxyethylamino)-1-methylbenzene, 1,3-diamino-2,4-dimethoxybenzene, 3,5-diamino-2-methoxy-toluene, 2-aminobenzoic acid, 3-aminobenzoic acid, 4-aminobenzoic acid, 2-aminophenylacetic acid, 3-aminophenylacetic acid, 4-aminophenylacetic acid, 2,3-diaminobenzoic acid, 2,4-diaminobenzoic acid, 2,5-diaminobenzoic acid, 3,4-diaminobenzoic acid, 3,5-diaminobenzoic acid, 4-aminosalicylic acid, 5-aminosalicylic acid, 3-amino-4-hydroxybenzoic acid, 4-amino-3-hydroxybenzoic acid, 2-aminobenzenesulfonic acid, 3-aminobenzenesulfonic acid, 4-aminobenzenesulfonic acid, 3-amino-4-hydroxybenzenesulfonic acid, 4-amino-3-hydroxynaphthalene-1-sulfonic acid, 6-amino-7-hydroxynaphthalene-2-sulfonic acid, 7-amino-4-hydroxynaphthalene-2-sulfonic acid, 4-amino-5-hydroxynaphthalene-2,7-disulfonic acid, 3-amino-2-naphthoic acid, 3-aminophthalic acid, 5-aminoisophthalic acid, 1,3,5-triaminobenzene, 1,2,4-triaminobenzene, 1,2,4,5-tetraaminobenzene, 2,4,5-triaminophenol, pentaaminobenzene, hexaaminobenzene, 2,4,6-triaminoresorcine, 4,5-diaminopyrocatechol, 4,6-diaminopyrogallol, 1-(2-hydroxy-5-aminobenzyl)-2-imidazolidinone, 4-amino-2((4-[(5-amino-2-hydroxyphenyl)methyl]-piperazinyl)methyl)phenol, 3,5-diamino-4-hydroxypyrocatechol, 1,4-bis-(4-aminophenyl)-1,4-diazacycloheptane, aromatic nitriles, such as 2-amino-4-hydroxybenzonitrile, 4-amino-2-hydroxybenzonitrile, 4-aminobenzonitrile, 2,4-diaminobenzonitrile, amino compounds with nitro groups, such as 3-amino-6-methylamino-2-nitropyridine, picramic acid, [8-[(4-amino-2-nitrophenyl)-azo]-7-hydroxy-naphth-2-yl]trimethylammonium chloride, [8-[(4-amino-3-nitrophenyl)-azo)-7-hydroxy-naphth-2-yl]trimethylammonium chloride (Basic Brown 17), 1-hydroxy-2-amino-4,6-dinitrobenzene, 1-amino-2-nitro-4-[bis(2-hydroxyethyl)amino]benzene, 1-amino-2-[(2-hydroxyethyl)amino]-5-nitrobenzene (HC Yellow Nr. 5), 1-amino-2-nitro-4-[(2-hydroxyethyl)amino]benzene (HC Red Nr. 7), 2-chloro-5-nitro-N-2-hydroxyethyl-1,4-phenylenediamine, 1-[(2-hydroxyethyl)amino]-2-nitro-4-aminobenzene (HC Red Nr. 3), 4-amino-3-nitrophenol, 4-amino-2-nitrophenol, 6-nitro-o-toluidine, 1-amino-3-methyl-4-[(2-hydroxyethyl)amino]-6-nitrobenzene (HC Violet Nr. 1), 1-amino-2-nitro-4-[(2,3-dihydroxypropyl)amino]-5-chlorobenzene (HC Red Nr. 10), 2-(4-amino-2-nitroanilino)benzoic acid, 6-nitro-2,5-diaminopyridine, 2-amino-6-chloro-4-nitrophenol, disodium salt of 1-amino-2-(3-nitrophenylazo)-7-phenylazo-8-naphthol-3,6-disulfonic acid (acid blue Nr.29), disodium salt of 1-amino-2-(2-hydroxy-4-nitrophenylazo)-8-naphthol-3,6-disulfonic acid (Palatinchrome green), disodium salt of 1-amino-2-(3-chloro-2-hydroxy-5-nitrophenylazo)-8-naphthol-3,6-disulfonic acid (Gallion), disodium salt of 4-amino-4′-nitrostilbene-2,2′-disulfonic acid, 2,4-diamino-3′,5′-dinitro-2′-hydroxy-5-methylazobenzene (Mordant brown 4), 4′-amino-4-nitrodiphenylamine-2-sulfonic acid, 4′-amino-3′-nitrobenzophenone-2-carboxylic acid, 1-amino-4-nitro-2-(2-nitrobenzylideneamino)benzene, 2-[2-(diethylamino)ethylamino]-5-nitroaniline, 3-amino-4-hydroxy-5-nitrobenzenesulfonic acid, 3-amino-3′-nitrobiphenyl, 3-amino-4-nitroacenaphthene, 2-amino-1-nitronaphthalene, 5-amino-6-nitrobenzo-1,3-dioxol, anilines, particularly anilines containing nitro groups, such as 4-nitroaniline, 2-nitroaniline, 1,4-diamino-2-nitrobenzene, 1,2-diamino-4-nitrobenzene, 1-amino-2-methyl-6-nitrobenzene, 4-nitro-1,3-phenylenediamine, 2-nitro-4-amino-1-(2-hydroxyethylamino)benzene, 2-nitro-1-amino-4-[bis(2-hydroxyethyl)amino]benzene, 4-amino-2-nitrodiphenylamine-2′-carboxylic acid, 1-amino-5-chloro-4-(2-hydroxyethylamino)-2-nitrobenzene, aromatic anilines or phenols with a further aromatic radical, as illustrated in Formula VII 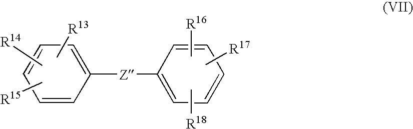
in which
R¹³ stands for a hydroxyl or an amino group, which can be substituted with a C₁-C₆ alkyl group, a C₁-C₆ hydroxyalkyl group, a C₁-C₆ alkoxy group or a C₁-C₆ alkoxy C₁-C₆ alkyl group,
R¹⁴, R¹⁵, R¹⁶, R¹⁷ and R¹⁸ independently of one another stand for a hydrogen atom, a hydroxyl or an amino group, which can be substituted with a C₁-C₆ alkyl group, a C₁-C₆ hydroxyalkyl group, a C₁-C₆ alkoxy group, a C₁-C₆ aminoalkyl group or a C₁-C₆ alkoxy C₁-C₆ alkyl group, and
Z″ is a direct bond, a saturated or unsaturated carbon chain with 1 to 4 carbon atoms, optionally substituted with hydroxyl groups, a carbonyl-, sulfonyl- or imino group, an oxygen- or sulfur atom, or a group with the Formula VIII
—Q′ [symbol] CH₂—Q—CH₂—Q [symbol] ₒ (VIII)
in which
Q means a direct bond, a CH₂— or CHOH group,
Q′ and Q″ independently of one another stand for an oxygen atom, an NR¹⁹ group, in which R¹⁹ means a hydrogen atom, a C₁-C₆ alkyl group or C₁-C₆ hydroxyalkyl group, wherein also both groups, together with the remainder of the molecule, can form a 5-, 6- or 7-membered ring, the groups O—(CH₂)ₚ—NH or NH—(CH₂)_{p′}—O, in which p and p′ are 2 or 3, and
means a number from 1 to 4,
such as, for example 4,4′-diaminostilbene and its hydrochloride, mono- or di-Na-salt of 4,4′-diaminostilbene-2,2′-disulfonic acid, 4-amino-4′-dimethylaminostilbene and its hydrochloride, 4,4′-diaminodiphenylmethane, 4,4′-diaminodiphenylsulfide, 4,4′-diaminodiphenylsulfoxide, 4,4′-diaminodiphenylamine, 4,4′-diaminodiphenylamine-2-sulfonic acid, 4,4′-diaminobenzophenone, 4,4′-diaminodiphenylether, 3,3′,4,4′-tetraaminodiphenyl, 3,3′,4,4′-tetraamino-benzophenone, 1,3-bis-(2,4-diaminophenoxy)propane, 1,8-bis-(2,5-diaminophenoxy)-3,6-dioxaoctane, 1,3-bis-(4-aminophenylamino)propane, 1,3-bis-(4-aminophenylamino)-2-propanol, 1,3-bis-[N-(4-aminophenyl)-2-hydroxyethylamino]-2-propanol, N,N-bis-[2-(4-aminophenoxy)ethyl]methyl amine, N-phenyl-1,4-phenylenediamine and bis(5-amino-2-hydroxyphenyl)methane.

11. The agent according to claim 9, characterized in that the aromatic hydroxyl compounds of component D are selected from the group consisting of 2-, 4-, 5-methylresorcinol, 2,5-dimethylresorcinol, resorcinol, 3-methoxyphenol, pyrocatechol, hydroquinone, pyrogallol, phloroglucinol, hydroxyhydroquinone, 2-, 3-, 4-methoxy-, 3-dimethylamino-, 2-(2-hydroxyethyl)-, 3,4-methylenedioxyphenol, 2,4-, 3,4-dihydroxybenzoic acid, -phenylacetic acid, gallic acid, 2,4,6-trihydroxybenzoic acid, -acetophenone, 2-, 4-chlororesorcinol, 1-naphthol, 1,5-, 2,3-, 2,7-dihydroxynaphthalene, 6-dimethylamino-4-hydroxy-2-naphthalenesulfonic acid and 3,6-dihydroxy-2,7-naphthalenesulfonic acid.

12. The agent according to claim 9, characterized in that the nitrogen-containing heterocyclic compounds of component D are selected from the group consisting of 2-aminopyridine, 3-aminopyridine, 4-aminopyridine, 2-amino-3-hydroxypyridine, 2,6-diaminopyridine, 2,5-diaminopyridine, 2-(aminoethylamino)-5-aminopyridine, 2,3-diaminopyridine, 2-dimethylamino-5-aminopyridine, 2-methylamino-3-amino-6-methoxypyridine, 2,3-diamino-6-methoxypyridine, 2,6-dimethoxy-3,5-diaminopyridine, 2,4,5-triaminopyridine, 2,6-dihydroxy-3,4-dimethylpyridine, N-[2-(2,4-diaminophenyl)aminoethyl]-N-(5-amino-2-pyridyl)amine, N-[2-(4-aminophenyl)aminoethyl]-N-(5-amino-2-pyridyl)amine, 2,4-dihydroxy-5,6-diaminopyrimidine, 4,5,6-triaminopyrimidine, 4-hydroxy-2,5,6-triaminopyrimidine, 2-hydroxy-4,5,6-triaminopyrimidine, 2,4,5,6-tetraaminopyrimidine, 2-methylamino-4,5,6-triaminopyrimidine, 2,4-diaminopyrimidine, 4,5-diaminopyrimidine, 2-amino-4-methoxy-6-methyl-pyrimidine, 3,5-diaminopyrazole, 3,5-diamino-1,2,4-triazole, 3-aminopyrazole, 3-amino-5-hydroxypyrazole, 1-phenyl-4,5-diaminopyrazole, 1-(2-hydroxyethyl)-4,5-diaminopyrazole, 1-phenyl-3-methyl-4,5-diaminopyrazole, 4-amino-2,3-dimethyl-1-phenyl-3-pyrazolin-5-one (4-aminoantipyrine), 1-phenyl-3-methylpyrazol-5-one, 2-aminoquinoline, 3-aminoquinoline, 8-aminoquinoline, 4-aminoquinaldine, 2-aminonicotinic acid, 6-aminonicotinic acid, 5-aminoisoquinoline, 5-aminoindazole, 6-aminoindazole, 5-aminobenzimidazole, 7-aminobenzimidazole, 5-aminobenzothiazole, 7-aminobenzothiazole, 2,5-dihydroxy-4-morpholino-aniline as well as indole- and indoline derivates, such as 4-aminoindole, 5-aminoindole, 6-aminoindole, 7-aminoindole, 5,6-dihydroxyindole, 5,6-dihydroxyindoline, 4-hydroxyindoline and hydroxypyrimidine derivatives and the physiologically acceptable salts of the abovementioned compounds.

13. The agent according to claim 9, characterized in that the amino acids are selected from arginine, histidine, tyrosine, phenylalanine, DOPA (dihydroxyphenylalanine), ornithine, proline, lysine, tryptophan, 6-aminocaproic acid and β-alanine.

14. The agent according to claim 9, characterized in that the component D is selected from at least one compound of the group consisting of N-(2-hydroxyethyl)-N-ethyl-p-phenylenediamine, 2-chloro-p-phenylenediamine, N,N-bis-(2-hydroxyethyl)-p-phenylenediamine, 2-aminophenol, 3-aminophenol, 4-aminophenol, 2-amino-6-chloro-4-nitrophenol, p-phenylenediamine, 2-(2,5-diaminophenyl)ethanol, 2,5-diaminotoluene, 3,4-methylenedioxyaniline, 2-amino-4-(2-hydroxyethylamino)anisole, 2-(2,4-diaminophenoxy)ethanol, 3-amino-2,4-dichlorophenol, 2-methyl-5-aminophenol, 3-methyl-4-aminophenol, 2-methyl-5-(2-hydroxyethylamino)phenol, 2-methyl-5-amino-4-chlorophenol, 6-methyl-3-amino-2-chlorophenol, 2-aminomethyl-4-aminophenol, 2-diethylaminomethyl-4-aminophenol, 2-dimethylaminomethyl-4-aminophenol, 2,6-dichloro-4-aminophenol, 2-hydroxymethyl-4-aminophenol, 2,6-bis(2-hydroxyethylamino)-1-methylbenzene, bis(2-hydroxy-5-aminophenyl)methane, bis-(4,5-amino-2-hydroxyphenyl)methane, 1,3-bis(2,4-diaminophenoxy)propane, 1,4-bis(4-aminophenyl)-1,4-diazacycloheptane, 1,8-bis(2,5-diaminophenoxy)-3,6-dioxaoctane, 4,4′-diaminodiphenylamine, 3,4-methylenedioxyphenol, 3,4-diaminobenzoic acid, 2,5-diaminopyridine, 2-dimethylamino-5-aminopyridine, 2-amino-3-hydroxypyridine, 3-amino-2-methylamino-6-methoxypyridine, 2,3-diamino-6-methoxypyridine, 3,5-diamino-2,6-dimethoxypyridine, 2,6-dihydroxy-3,4-dimethylpyridine, 2-hydroxy-4,5,6-triaminopyrimidine, 4-hydroxy-2,5,6-triaminopyrimidine, 2,4,5,6-tetraaminopyrimidine, 2-methylamino-4,5,6-triamino-pyrimidine, 3,5-diaminopyrazole, 3-amino-5-hydroxypyrazole, 4,5-diamino-1-(2-hydroxyethyl)pyrazole, 5,6-dihydroxyindole, 5,6-dihydroxyindoline, 4-amino-2,3-dimethyl-1-phenyl-3-pyrazolin-5-one (4-aminoantipyrine), β-alanine, L-proline, L-lysine, DL-tyrosine as well as their physiologically acceptable salts.

15. The dyeing agent according to claim 9, characterized in that the compounds of component D are comprised in an amount of 0.03 to 65 mmol, based on 100 g of the total dyeing agent.

16. The dyeing agent according to claim 1, characterized in that the compounds of component B and the compounds of component C are each comprised in an amount of 0.03 to 65 mmol, based on 100 g of the total dyeing agent.

17. The agent according to claim 1, characterized in that the agent further comprises a color enhancer selected from the group consisting of piperidine, piperidine-2-carboxylic acid, piperidine-3-carboxylic acid, piperidine-4-carboxylic acid, pyridine, 2-hydroxypyridine, 3-hydroxypyridine, 4-hydroxypyridine, imidazole, 1-methylimidazole, arginine, histidine, pyrrolidine, proline, pyrrolidone, pyrrolidone-5-carboxylic acid, pyrazole, 1,2,4-triazole, piperazidine, methoxybutanol, propylene carbonate, ethylene carbonate, their derivatives and physiologically acceptable salts and mixtures thereof.

18. The agent according to claim 1, characterized in that the agent further comprises at least one substantive dye, preferably in an amount from 0.01 to 20 wt. %, based on the components in addition to the components used according to the invention.

19. The agent according to claim 1, characterized in that the agent further comprises an oxidation dye precursor which consists essentially of at least one developer component and optionally at least one coupler component.

20. The agent according to claim 1, characterized in that the agent further comprises salts formed from anions selected from the group of formates, carbonates, halides, sulfates, butyrates, valeriates, capronates, acetates, lactates, glycolates, tartrates, citrates, gluconates, propionates, phosphates and phosphonates and cations selected from the group consisting of ammonium, potassium, sodium, lithium, magnesium, calcium, strontium, barium, aluminum, manganese, iron, cobalt, copper and zinc.

21. The agent according to claim 1, characterized in that the agent further comprises at least one surfactant selected from the group consisting of anionic, zwitterionic and nonionic surfactants.

22. The agent according to claim 1, characterized in that the pH of the agent is between 2 and 12.

23. A process of dyeing keratin-containing fibers, said process comprising the step of applying to the fibers the agent of claim 1.

24. The process for dyeing keratin-containing fibers according to claim 23, wherein the keratin-containing fibers consist of human hair, said process comprising the steps of applying the agent to hair for a period of about 15 to about 30 minutes, followed by a step of removing at least some of the agent by rinsing the hair with water or washing the hair with shampoo.

25. The process according to claim 23, characterized in that in the step of applying to the fibers the agent of claim 1, components B and C are applied separately.

26. The process according to claim 23, characterized in that in the step of applying to the fibers the agent of claim 1, components A, B and C are applied separately.
